(19) United States
(12) Patent Application Publication
(10) Pub. No.: US 2023/0207141 A1
(43) Pub. Date: Jun. 29, 2023

(54) METHOD AND SYSTEM FOR PATIENT ENGAGEMENT

(71) Applicant: Shwetha Padma Bharadwaj, Los Altos, CA (US)

(72) Inventor: Shwetha Padma Bharadwaj, Los Altos, CA (US)

(21) Appl. No.: 18/090,417

(22) Filed: Dec. 28, 2022

Related U.S. Application Data
(60) Provisional application No. 63/294,790, filed on Dec. 29, 2021.

(51) Int. Cl. G16H 80/00; G16H 50/30

(52) U.S. Cl. CPC G16H 80/00; G16H 50/30

(57) ABSTRACT

Embodiments herein provide a method for a patient to increase engagement with cognitive behavioral therapy by a system (100), which in turn will increase the efficacy of the therapy. The embodiment is meant to be used in a clinical setting, specifically in an outpatient group program with a supervising therapist that meets weekly, bi-weekly, or monthly. The embodiment uses machine learning (ML), a group component, and gamification to promote increased engagement. ML (natural language processing) is used to help the patient automatically classify his/her negative thoughts into a pre-set taxonomy of ‘cognitive distortions’ or retrieve a previously stored triple column entry. The group component allows for the patient to request help from his/her groupmates in between in-person meetings. Gamification provides an additional incent to participate in the therapy: Within groups, teams of three patients compete against each other by scoring points proportional to their engagement with the app.

### PRIORITY DETAILS

The present application is based on, and claims priority from, U.S. Application No. 63/294,790, filed on 29, December 2021, the disclosure of which is hereby incorporated by reference herein.

### TECHNICAL FIELD

This embodiment relates to patient engagement, and more particularly, to a method and a system for leveraging Machine Learning (ML) models, crowdsourcing, and gamification to increase patient engagement.

### BACKGROUND

Cognitive Behavioral Therapy (CBT) is a form of psychological treatment used to treat problems including depression, anxiety disorders, alcohol and drug use problems, marital problems, eating disorders, and severe mental illness. According to American Psychological Association, numerous research studies suggest that CBT leads to significant improvement in functioning and quality of life. In many studies, CBT has been demonstrated to be as effective as, or more effective than, other forms of psychological therapy or psychiatric medications.

CBT involves helping a patient to navigate and combat recurrent and entrenched negative thoughts with logical repudiations. These negative thoughts are referred to as Automatic Negative Thoughts (ANTs). The premise behind CBT is that these thoughts are unsound, and only arise in an otherwise logical person because of the nature of a mood disorder. CBT hypothesizes that these thoughts are logically fallacious but perceived as valid (even by those around the patient including the treating therapist) because of a sense of self-worthlessness by the patient. Finally, CBT proposes that these thoughts are cognitive distortions and can be classified into a taxonomy.

Once compartmentalized into a concrete label, specific arguments can be used to invalidate them and lend the bearer a sense of clarity to address the underlying problem. For example, for the thought, or self-criticism ‘You are such a loser because you failed this test,’ one could assign the cognitive distortion label of ‘overgeneralization’ (i.e. the patient perceives a single negative event as a negative ending pattern of defeat.) or ‘all-or-nothing thinking’ (i.e., the patient perceives things in black and white categories. If a patient's performance falls short of a perfect outcome, the patient considers himself/herself a total failure). According to CBT, one can repartee these thoughts in the self-defense column. For example, ‘Just because you failed one test doesn't make you a loser.’

For CBT to be effective, the patient must be able to articulate their state of mind that can be mapped to one or more known cognitive distortion labels. Further, the patient must also be able to choose a self-activation activity that helps the patient to break the lethargy cycle. However, the patient can find it challenging to navigate the process of identifying thoughts and following up on self-activation activities. As a result, the patient slowly disengages and eventually discontinues the treatment.

Therefore, there is a need for a system that provides a way to keep the patient engaged in the treatment.

### SUMMARY

The present disclosure provides a patient engagement method. The method comprises receiving, by a patient engagement system, an input from a first patient from a plurality of patients, where the input indicates that the first patient has an Automatic Negative Thought (ANT). Further, the method comprises displaying, by the patient engagement system, a first option to participate in a triple-column associated with the ANT of the first patient, a second option for assistance of a second patient of the plurality of patients, and a third option for assistance of an application. Further, the method comprises detecting, by the patient engagement system, the first option, or the second option or the third option selected by the first patient. The method comprises when the first option is selected by the first patient, displaying the triple-column, receiving an input consisting of the ANT, a cognitive distortion and a rational response in the triple-column from the first patient and allocating a score to the first patient based on the input provided by the first patient. The method comprises when the second option is selected by the first patient, sending a cognitive distortion and rational response request is sent to the second patient. After it's been answered, the first patient receives the cognitive distortion and the rational response for the ANT from the second patient, and the system allocates a score to the second patient and the first patient upon acceptance of the first patient of the cognitive distortion and the rational response for the ANT received from the second patient. If the third option is selected by the first patient, the application predicts the cognitive distortion and the rational response for the ANT using a Machine Learning (ML) model associated with the application, and allocating a score to the first patient upon acceptance of the first patient for the cognitive distortion and the rational response for the ANT predicted by the ML model.

In an embodiment, allocating the score to the second patient and the first patient upon acceptance of the first patient for the cognitive distortion and the rational response for the ANT received from the second patient comprises adding the score to a previously allocated score of the second patient, and the first patient upon acceptance of the first patient for the cognitive distortion and the rational response for the ANT of the first patient received from the second patient as well as incrementing the score of the team that the second and first patients belong to.

In an embodiment, allocating the score to the first patient upon acceptance of the first patient for the cognitive distortion and the rational response for the ANT predicted by the ML model comprises adding the score to a previously allocated score of the first patient upon acceptance of the first patient for the cognitive distortion and the rational response predicted by the ML model for the ANT of the first patient as well as incrementing the score of the team that the first patient belongs to.

In an embodiment, sending a cognitive distortion and rational response request to the second patient comprises determining whether a crowd stipend of the first patient meets a crowd stipend threshold; sending the cognitive distortion and rational response request to the second patient when the crowd stipend of the first patient meets the crowd stipend threshold; and decrementing the crowd stipend of the first patient.

In an embodiment, receiving, by the patient engagement system, the cognitive distortion and the rational response for the ANT from the second patient, comprises receiving, by the patient engagement system, the cognitive distortion for the ANT and a rationale for selecting the cognitive distortion from the second patient; displaying, by the patient engagement system, an untwist-your-thinking strategy activity to be performed by the second patient based on the cognitive distortion when only the cognitive distortion for the ANT and a rationale for selecting the cognitive distortion is received from the second patient, but not a rational response, and should the patient choose to engage with the Untwist Your Thinking Strategy activity; and receiving, by the patient engagement system, the rational response for the ANT upon performing the untwist-your-thinking strategy by the second patient.

In an embodiment, acceptance of the first patient of the cognitive distortion and the rational response of the ANT received from the second patient or predicted by the ML model comprises sending, by the patient engagement system, the cognitive distortion and the rational response for the ANT to the first patient; receiving, by the patient engagement system, an input indicating acceptance of the cognitive distortion and rejection of the rational response by the first patient; displaying, by the patient engagement system, an untwist-your-thinking strategy activity to be performed by the first patient based on the input and the cognitive distortion; and receiving, by the patient engagement system, the rational response for the ANT upon performing the untwist-your-thinking strategy by the first patient.

In an embodiment, the method comprises receiving, by the patient engagement system, the score allocated to each patient of the plurality of patients, where the plurality of patients is associated with a group. Further, the method comprises assigning, by the patient engagement system, a score to the group by combining the score of each patient of the plurality of patients associated with the group. Further, the method comprises storing, by the patient engagement system, the score of the group and the score allocated to each patient of the plurality of patients in the patient engagement system into a memory. Further, the method comprises displaying, by the patient engagement system, the score and a rank of each group.

In an embodiment, the method includes allocating, by the patient engagement system, a score to the first patient and the group proportional to an engagement of the first patient with the patient engagement system, and a therapist of the first patient.

In an embodiment, the method includes detecting, by the patient engagement system, a rejection of the first patient on the cognitive distortion and the rational response for the ANT received from the ML model or the at least one second patient. Further, the method includes storing by the patient engagement system, the ANT, to a problematic thoughts database.

In an embodiment, the method includes displaying, by the patient engagement system, the ANT in the problematic thoughts database to the therapist during the in-person group therapy session or based on a user input. Further, the method includes receiving cognitive distortion and the rational response for the ANT from the therapist.

In an embodiment, the method comprises storing, by the patient engagement system, the cognitive distortion and the rational response for the ANT into the memory.

In an embodiment, predicting the cognitive distortion and the rational response for the ANT using the ML model associated with the application comprises retrieving, by the patient engagement system, a previously stored ANT similar to the ANT using the ML model based on semantic similarity that the particular patient has stored, and a cognitive distortion and a rational response of the previously stored ANT from the memory.

In an embodiment, predicting the cognitive distortion and the rational response for the ANT using the ML model associated with the application comprises assigning, by the patient engagement system, a cognitive distortion for an unrecognized ANT.

The present disclosure provided the patient engagement system. The patient engagement system includes the memory and a processor coupled to the memory, where the memory comprises information of the plurality of patients. The processor receives the input from the first patient from the plurality of patients, where the input indicates that the first patient has the ANT. The processor displays the first option to participate in the triple-column associated with the ANT of the first patient, the second option for the assistance of the second patient of the plurality of patients, and the third option for the assistance of the application. The processor detects the first option, the second option or the third option selected by the first patient. When the first option is selected by the first patient, the processor displays the triple-column. Further, the processor receives the input corresponding to the ANT, a cognitive distortion and a rational response in the triple-column from the first patient. Further, the processor allocates the score to the first patient based on the input provided by the first patient and a score to the first patient's team. When the second option is selected by the first patient, the processor sends the cognitive distortion and the rational response request to the second patient who is a part of the first patient's team. Further, the processor receives cognitive distortion and the rational response for the ANT from the second patient. Further, the processor allocates the score to the second patient and the first patient upon acceptance of the first patient for the cognitive distortion and the rational response for the ANT received from the second patient and the team they are in. When the third option is selected by the first patient, the processor predicts the cognitive distortion and the rational response for the ANT using the ML model associated with the application. Further, the processor allocates the score to the first patient upon acceptance of the first patient for the cognitive distortion and the rational response for the ANT predicted by the ML model along with incrementing score of the team the first patient is a member of.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments, and the embodiments herein include all such modifications.

### BRIEF DESCRIPTION OF FIGURES

This embodiment is illustrated in the accompanying drawings, throughout which like reference letters indicate corresponding parts in the various figures. The embodiments herein will be better understood from the following description with reference to the drawings, in which:

FIG. 1 is a block diagram illustrating a system for enabling patient engagement in psychotherapy, according to an embodiment herein;

FIGS. 2A to 2F constitute a flow diagram illustrating a method for classifying ANTs using automation and crowd-sourcing help from trusted people, according to an embodiment herein;

FIGS. 3A to 3B constitute a flow diagram illustrating a method for patient engagement through self-activation activities and daily activity schedule, according to an embodiment herein;

FIG. 4 is a block diagram illustrating a computing environment for an application server enabling the methods disclosed herein, according to an embodiment herein;

FIG. 5 is a block diagram of a patient engagement system for the patient engagement, according to another embodiment herein;

FIG. 6 is a flow chart illustrating a patient engagement method, according to embodiments as disclosed herein;

FIG. 7 is a flow chart illustrating a method for signing up the patient to the patient engagement system, according to embodiments as disclosed herein;

FIG. 8 illustrates an example scenario of providing a triple-column to a patient by the patient engagement system, according to embodiments as disclosed herein;

FIGS. 9A to 9B constitute a flow chart illustrating an overall flow of the patient engagement method, according to embodiments as disclosed herein;

FIGS. 10A to 10C illustrate an example scenario of assisting with an application or a teammate to a first patient for providing a cognitive distortion, and a rational response of ANT of the patient, according to embodiments as disclosed herein;

FIG. 11 is a flow chart illustrating a method of consuming crowd stipend, according to embodiments as disclosed herein;

FIG. 12 is a flow chart illustrating a method of handling the crowd stipend and providing the cognitive distortion and the rational response to the first patient, according to embodiments as disclosed herein;

FIG. 13 illustrates an example scenario of obtaining the assistance of a teammate for resolving the cognitive distortion of the rational response of the ANT of the first patient, according to embodiments as disclosed herein;

FIGS. 14A to 14B constitute a flow chart illustrating a method of handling the crowd stipend and providing the cognitive distortion and the rational response to the first patient, according to embodiments as disclosed herein;

FIGS. 15A to 15C illustrate an example scenario of handling the cognitive distortion and/or the rational response provided by a second patient for the ANT of the first patient, according to embodiments as disclosed herein;

FIGS. 16A to 16B constitute a flow chart illustrating a method of handling the cognitive distortion and/or the rational response provided by the second patient for the ANT of the first patient, according to embodiments as disclosed herein;

FIGS. 17A to 17C illustrates an example scenario of handling the problematic ANT by the patient engagement system, according to embodiments as disclosed herein;

FIGS. 18A to 18B constitute a flow chart illustrating a method of the problematic ANT by the system, according to embodiments as disclosed herein; and

FIGS. 19A to 19D illustrate an example scenario of displaying a leaderboard, and an admin portal by the patient engagement system, according to embodiments as disclosed herein.

### DETAILED DESCRIPTION OF EMBODIMENT

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

As is traditional in the field, embodiments may be described and illustrated in terms of blocks which carry out a described function or functions. These blocks, which may be referred to herein as managers, units, modules, hardware components or the like, are physically implemented by analog and/or digital circuits such as logic gates, integrated circuits, microprocessors, microcontrollers, memory circuits, passive electronic components, active electronic components, optical components, hardwired circuits and the like, and may optionally be driven by firmware. Each block of the embodiments may be physically separated into two or more interacting and discrete blocks without departing from the scope of the disclosure. Likewise, the blocks of the embodiments may be physically combined into more complex blocks without departing from the scope of the disclosure.

The accompanying drawings are used to help easily understand various technical features and it should be understood that the embodiments presented herein are not limited by the accompanying drawings. As such, the present disclosure should be construed to extend to any alterations, equivalents and substitutes in addition to those which are particularly set out in the accompanying drawings. Although the terms first, second, etc. may be used herein to describe various elements, these elements should not be limited by these terms. These terms are generally only used to distinguish one element from another.

The principal object of this embodiment is to provide a patient engagement method and a patient engagement system.

The present disclosure provided a patient engagement method. The method comprises receiving, by a patient engagement system, an input from a first patient from a plurality of patients, where the input indicates that the first patient has an Automatic Negative Thought (ANT). Further, the method comprises displaying, by the patient engagement system, a first option to participate in a triple-column associated with the ANT of the first patient, a second option for assistance of a second patient of the plurality of patients, and a third option for assistance of an application. Further, the method comprises detecting, by the patient engagement system, the first option, or the second option or the third option selected by the first patient. The method comprises when the first option is selected by the first patient, displaying the triple-column in the triple-column mode, receiving an input corresponding the ANT, a cognitive distortion and a rational response in the triple-column from the first patient and allocating a score to the first patient based on the input provided by the first patient. The method comprises when the second option is selected by the first patient, sending a cognitive distortion and rational response request to the second patient, receiving the cognitive distortion and the rational response for the ANT from the second patient, and allocating a score to the second patient and the first patient upon acceptance of the first patient for the cognitive distortion and the rational response for the ANT received from the second patient. The method comprises when the third option is selected by the first patient, predicting the cognitive distortion and the rational response for the ANT using a Machine Learning (ML) model associated with the application, and allocating a score to the first patient upon acceptance of the first patient for the cognitive distortion and the rational response for the ANT predicted by the ML model.

The present disclosure provided the patient engagement system. The patient engagement system includes the memory and a processor coupled to the memory, where the memory comprises information of the plurality of patients. The processor receives the input from the first patient from the plurality of patients, where the input indicates that the first patient has the ANT. The processor displays the first option to participate in the triple-column associated with the ANT of the first patient, the second option for assistance of the second patient of the plurality of patients, and the third option for assistance of the application. The processor detects the first option, the second option or the third option selected by the first patient. When the first option is selected by the first patient, the processor displays the triple-column. Further, the processor receives the input corresponding to the ANT, the cognitive distortion and the rational response in the triple-column from the first patient. Further, the processor allocates the score to the first patient based on the input provided by the first patient. When the second option is selected by the first patient, the processor sends the cognitive distortion and the rational response request to the second patient. Further, the processor receives cognitive distortion and the rational response for the ANT from the second patient. Further, the processor allocates the score to the second patient and the first patient upon acceptance of the first patient for the cognitive distortion and the rational response for the ANT received from the second patient. When the third option is selected by the first patient, the processor predicts the cognitive distortion and the rational response for the ANT using the ML model associated with the application. Further, the processor allocates the score to the first patient upon acceptance of the first patient for the cognitive distortion and the rational response for the ANT predicted by the ML model.

The embodiments herein disclose a system for enabling methods to increase patient engagement in psychotherapy. Embodiments enable patients to create a schedule of activities and incentivize patients to be engaged in the activities through notifications, gamification, and crowd-sourced inputs. The methods allow patients to enter negative thought (ANT) into the system, classify the thoughts into known distortion categories, and provide a rational response (also referred to as “rebuttal”) to the negative thought.

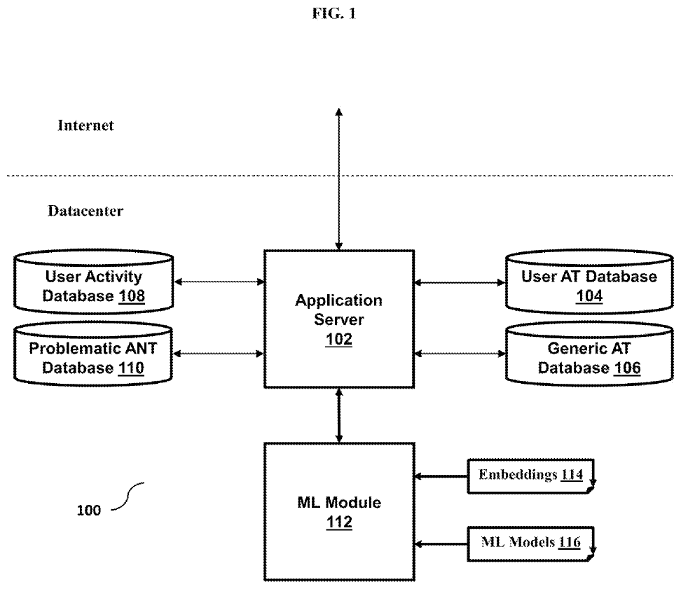

Referring now to the drawings, and more particularly to FIGS. 1 through 19, where similar reference characters denote corresponding features consistently throughout the figures, there are shown embodiments.

FIG. 1 is a block diagram illustrating a patient engagement system (100) for enabling patient engagement in psychotherapy, according to a preferred embodiment. The patient engagement system (100) includes an application server (102) hosted in a data center. The application server (102) hosts the business logic to perform various methods disclosed herein. In addition to the business logic, the application server (102) can also host a web application for users of the patient engagement system (100) to engage with the application server (102) (for example, using a web browser), and can also host necessary Application programmable Interfaces (APIs) to interact with the application server (102) through a variety of other applications enabling user interfaces, including but not limited to mobile applications (for example, IOS, ANDROID etc.), and native applications for specific platforms such as WINDOWS, MAC OS and so on. The users of the patient engagement system (100) are a plurality of patients.

The various methods outlined in this section are enabled by an application server (102) (102), which is part of a patient engagement system (100). The primary interaction for various actions performed by users (a patient (first patient) and other members (second patients)) of the patient engagement system (100) is with the application server (102) (102).

The application server (102) is connected to (for example, over a local area network within a data center) at least one user thought database (104) (also referred to as “user AT database” or “user specific database”) to store user information including but not limited to user profile information, user specific preferences including but not limited to user negative thoughts (also called as ANT) and related classification information and any associated rationale, information related to friends and family to users involved in the process of identifying the classification of negative thoughts, etc.

The application server (102) is also connected to (for example, over a local area network within a data center) to at least one generalized database (106) (also referred to as “user AT database” or “generic database”) for storing negative thoughts, associated classification information, and any rationale associated with negative thoughts. The generalized database stores anonymized information as opposed to the user-specific database (104).

The application server (102) is also connected to a user activity database (108). The user activity database (108) stores information about patient activities, the status of activities taken up by the patients, and related master/pleasure levels. The information stored in the user activity database (108) can be used to track patient progress in relation to their schedule.

The application server (102) is also connected to a problematic ANT database (110). The problematic ANT database (110) can be used to store example negative thoughts to be used for negative model training. In a preferred embodiment, the problematic ANT database (110) is a static database. However, in some embodiments, the problematic ANT database (110) can also be used to store list of ANTs for which the patient is unable to find a rebuttal. The patient can use the personalized list to discuss with the patient's therapist at a later point in time. The patient engagement system (100) can use personalized unresolved ANTs for personalized model training. In some other embodiments, the list of personalized and unresolved ANTs can be stored in a separate database dedicated to that purpose.

In various embodiments, the information stored in the user specific database (104), the generic database (106), the user activity database (108), and the problematic ANT database (110) can be logical databases co-located within a single database instance. In some other embodiments, the database tables from the various databases depicted in FIG. 1 can also be co-located within in the single logical database.

The application server (102) is also connected to a Machine Learning (ML) module (112). The ML module (112) is responsible for automatically classifying the negative thoughts based on a pre-configured taxonomy. In an example embodiment, the taxonomy can be based on cognitive distortion labels identified by Dr. David D. Burns (listed in Table 1).

TABLE 1
Cognitive distortions

| Distortion | Description |
|---|---|
| All-or-nothing thinking | You think things in absolute, |
| | black-and-white categories |
| Overgeneralization | You look at a negative event as a |
| | never-ending pattern of defeat |
| Mental filter | You dwell on the negatives |
| Discounting the positives | You insist that your accomplishments |
| | or positive qualities do not count |
| Jumping to conclusions: | You regard others' response as |
| mind-reading | negative to you but there is no |
| | exact evidence |
| Jumping to conclusions: | You arbitrarily predict the things |
| fortune-telling | will turn out badly |
| Magnification or | You blow things way out of proportion, |
| minimization | or you shrink their importance |
| Emotional reasoning | You reason from how you feel: “I feel |
| | like an idiot, so I really must be one.” |
| Should statement | You criticize yourself (or other people) |
| | with “should,” “ought,” “must” |
| | and “have to.” |
| Labeling | Instead of saying “I made a mistake,” |
| | you tell yourself, “I'm a jerk,” or |
| | “a fool,” or “a loser.” |
| Personalization | You blame yourself for something |
| and blame | you were not entirely responsible |
| | for, or you blame others |

The ML module (112) can use one or more ML models (116), and one or more pre-learned embeddings 114 for determining the similarity of the negative thoughts to one or more pre-defined labels or classifications. In addition, the ML module (112) can use one or more negative models to minimize wrong classifications. The negative models can be trained using examples in the problematic ANT database (110). In a preferred embodiment, the negative model training can use the same model as the one used for positive model training but using a different loss function suitable for negative training.

In a preferred embodiment, the ML module (112) can use Sentence-Bidirectional Encoder Representations from Transformers (S-BERT) based embeddings that enable finding similar sentences in a computationally efficient manner. The S-BERT-based embeddings can be generated for both user-specific datasets and anonymized datasets.

Further, the ML module (112) can use a cross-encoder (for example, BERT trained) for the purposes of classification.

The ML module (112) can obtain data stored in any of the databases connected to the application server (102) via Application Programmable Interfaces (APIs). The APIs can be exposed as Restitutional State Transfer (REST) APIs on HTTP, or they can be exposed via another transport such as Google Remote Procedure Calls (gRPC).

In an embodiment, the patient engagement system (100) can be deployed in a data center (on premises, or in the cloud) where the application server (102), the user database (104), the generic database (106), the user activity database (108), the problematic ANT database (110), the ML module (112) and associated components are all co-located within a local area network. In other embodiments, the patient engagement system (100) can be deployed across environments (combination of on premises and cloud), where various components of the patient engagement system (100) are distributed across on-premises patient engagement system (100) and the cloud and are connected over a wide area network.

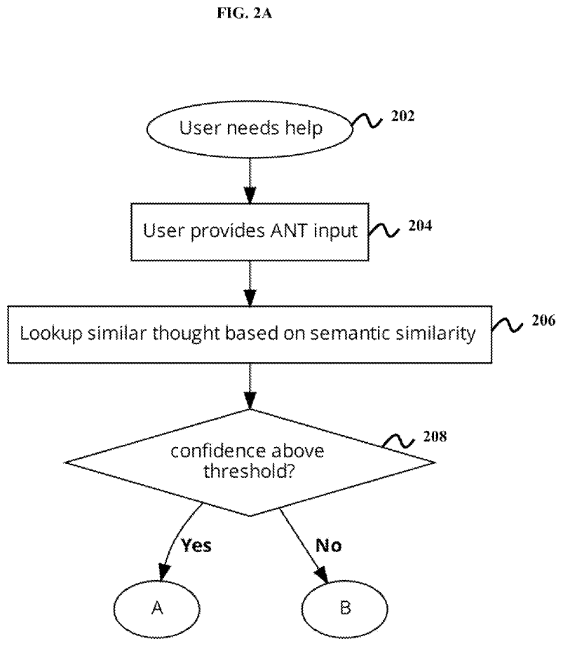

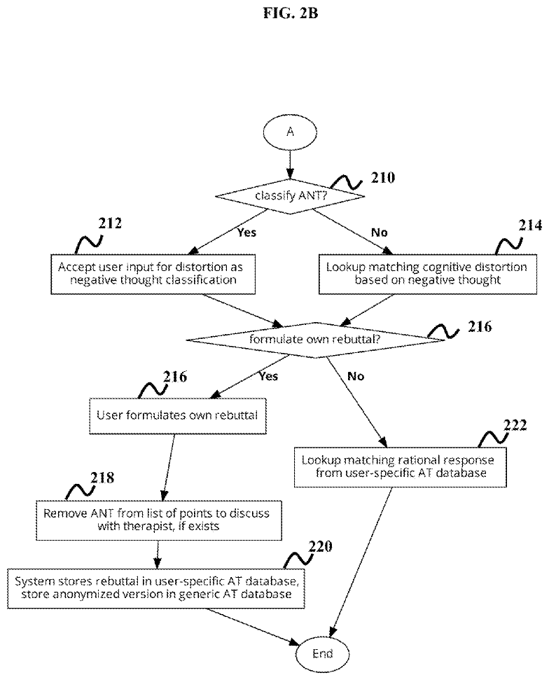

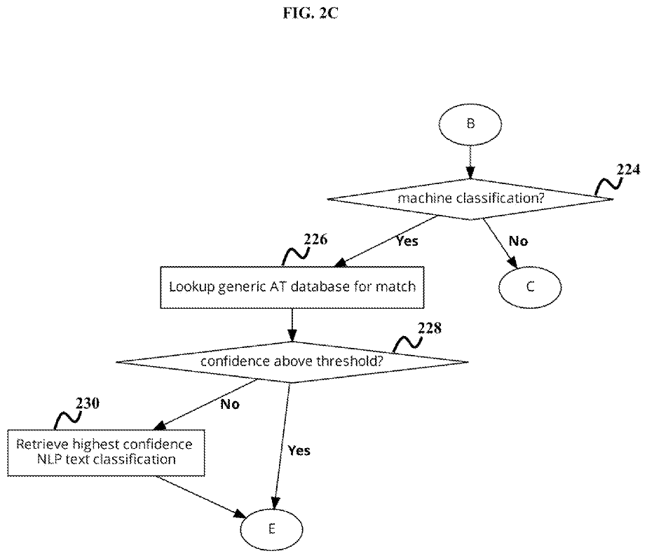

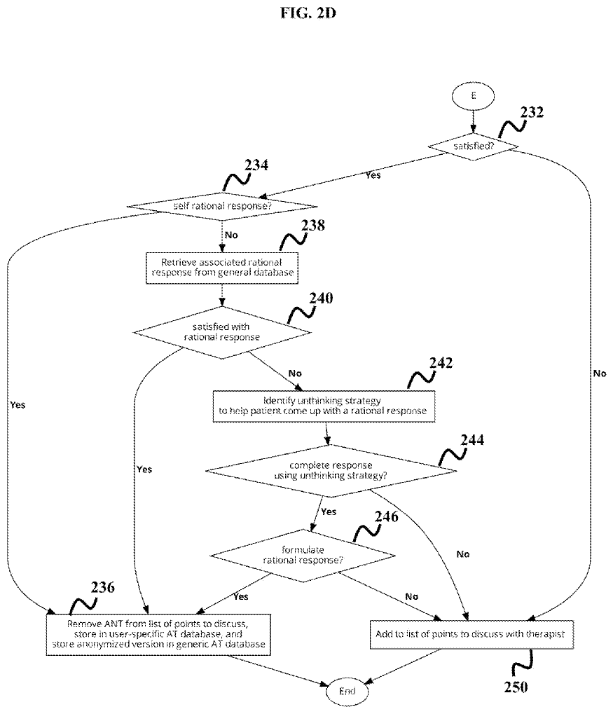

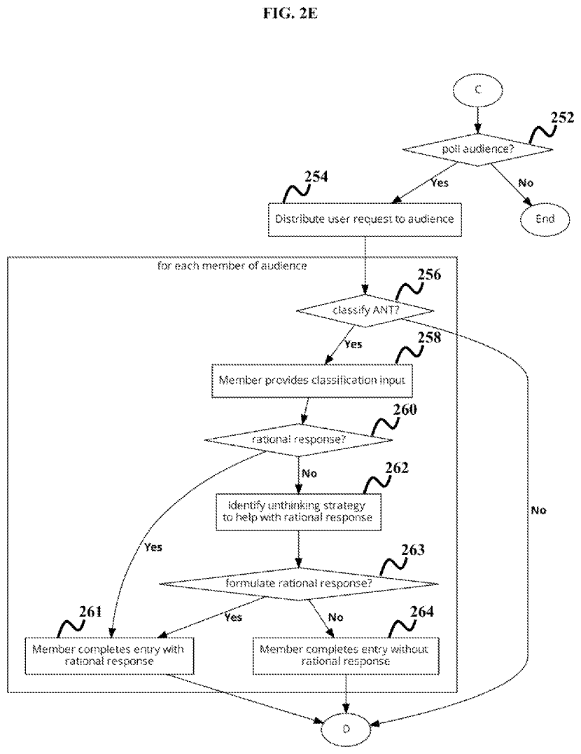

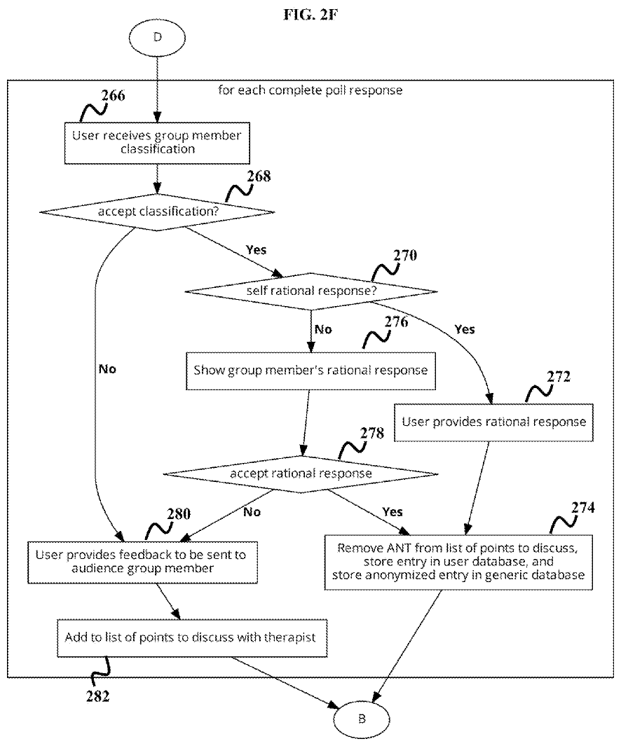

FIG. 2A to FIG. 2F (collectively referred to as FIG. 2) illustrate a method for classifying ANTs using automation and crowd-sourcing help from trusted people, according to a preferred embodiment.

A patient's interaction can start with one or more negative thoughts (202). The patient enters (204) the negative thought as an input (for example, using a mobile application). The server looks up (206) for similar thoughts that have associated rationale that can be used as the rebuttal to the negative thought. In a preferred embodiment, similar thoughts can be identified using a suitable ML model (for example, S-BERT embedding/model schema for information retrieval coupled with algorithms to find nearest neighbors, such as Hierarchical Navigable Small Worlds—HNSW). In a preferred embodiment, each similarity computation (similarity score) is also associated with a corresponding confidence score.

After looking up similar thoughts along with associated confidence scores, the application server checks (208) for and identifies similar thoughts that cross a pre-defined threshold. If such similar thoughts exist, the server can identify related distortion and provide a rational response to the patient's negative thought, the rational response being the one associated with a similar thought from the user-specific database with the highest similarity score.

In a preferred embodiment, the server checks (210) if the patient would like to classify the negative thought into the distortion category. If the patient is willing to do so, a patient input (212) for the classification is accepted. If the patient is not willing to classify the negative thought, then the server looks up (214) the distortion category for the negative thought. The distortion category can be the category associated with a similar thought that was found by the server.

Further, the server checks with the patient if the patient would like to formulate (216) the patient's own rebuttal to the negative thought. This is to allow the patient to engage and analyze the patient's own thoughts. If the patient chooses to formulate the patient's own rebuttal, then the patient provides (216) the rebuttal as input. The server checks if the negative thought is part of list of points/issues to be discussed with the therapist. If so, the server removes (218) the negative thought from the list, as the negative thought is now considered resolved after the patient can provide the rebuttal. The server stores (220) such rebuttals in the user-specific database for future reference. Further, the server also stores anonymized versions in the generic database. In a preferred embodiment, steps 218 and 220 are performed simultaneously, as part of a single step by a machine.

If the patient chooses not to provide the patient's own rebuttal to the negative thought, the server looks up (222) available rational responses from the user-specific database, based on responses stored against similar negative thoughts. And server chooses the rational response recorded against a similar thought with the highest score compared to other similar thoughts.

### Automatic Classification of Patient's ANT

In the case, the server is not able to find any similar thoughts that cross the pre-defined threshold, the server checks (224) with the patient if the patient is interested in an automated machine classification of the negative thought. If the patient chooses to receive a machine-generated classification, the server looks (226) for similar thoughts in the generic database and the classification is recorded against similar thoughts.

If the server can find matching similar thoughts (having confidence above a pre-configured threshold) (228), the matching similar thought with the highest confidence is presented to the patient.

However, if the server is unable to find a matching similar thought using word embeddings for negative thoughts and therefore unable to find relevant classification, the server can try to find (230) matching similar thought(s) in the generic database using Natural Language Processing (NLP) based similarity. If a similar thought crosses a pre-configured threshold, the classification associated with such a similar thought can be presented to the patient.

When the patient is satisfied (232) with the classification presented, either using similarity-based matching or NLP-based text analysis, the patient can choose (234) to provide the patient's own rational response. If the patient chooses to provide a rational response, then the response is stored (236) in the user database. The corresponding anonymized version is stored (236) in the generic database. Further, the corresponding negative thought is removed (236) from the list of points/issues to be discussed with the therapist as the negative thought can now be considered resolved. However, if the patient chooses not to provide own rational response, then the patient engagement system (100) can assist the patient by retrieving (238) associated rational responses from the generic database if the classification resulted from similarity matching (226).

After reviewing the rational response from the generic database, the patient can choose to complete the response process by accepting the rational response if he/she is satisfied (240) with the rational response.

However, if the patient is not satisfied with the rational response or if the classification is a result of NLP-based text analysis, the server can assist the patient by identifying (242) an unthinking strategy (also referred to as “untwist thinking strategy”) using a pre-configured logical data structure that allows looking up the right unthinking strategy for a given negative thought and the distortion previously identified.

The patient can review and choose (244) to complete the rational response using the input from the unthinking strategy input. If the patient chooses to do so, the patient can choose (246) to further formulate the response and provide the rational response as an input. The rational response is then stored in the user-specific database. The corresponding anonymized version is stored in the generic database. Further, the negative thought is removed from the list of points/issues to be discussed with the therapist, if present.

If the patient is not able to formulate the rational response after reviewing the unthinking strategy or if the patient is unable to formulate their own rational response, the patient engagement system (100) can aid the patient in adding (250) the negative thought to a list of points or issues to be discussed with a therapist.

Table 2 provides a list of untwist thinking strategies as illustrative examples, from the book, “The Feeling Good Handbook” by David D. Burns.

TABLE 2
Ten ways to untwist-your-thinking

| Name | Description |
|---|---|
| Counter the | Write down your negative thoughts so you |
| Distortion | can see which of the cognitive distortions |
| | you're involved in. This will make it easier |
| | to think about the problem in a more |
| | positive and realistic way. |
| Examine the | Instead of assuming that your negative |
| Evidence | thought is true, examine the actual |
| | evidence for it. For example, if you feel |
| | that you never do anything right, you could |
| | list several things you have done |
| | successfully. |
| The Double-standard | Instead of putting yourself down in a harsh, |
| Method | condemning way, talk to yourself in the |
| | same compassionate way you would talk |
| | to a friend with a similar problem. |
| The Experimental | Do an experiment to test the validity of |
| Technique | your negative thoughts. For example, if, |
| | during an episode of panic, you become |
| | terrified that you're about to die of a heart |
| | attack, you could jog or run up and down |
| | several flights of stairs. This will prove that |
| | your heart is healthy and strong. |
| Thinking in Shades | Although this method might sound drab, |
| of Gray | the effects can be illuminating. Instead of |
| | thinking about your problems in |
| | all-or-nothing extremes, evaluate things on |
| | a range from 1 to 100. When things don't |
| | work out as well as you hoped, think about |
| | the experience as a partial success rather |
| | than a complete failure. See what you can |
| | learn from the situation. |
| The Survey | Ask people questions to find out if your |
| Method | thoughts and attitudes are realistic. For |
| | example, if you believe that public |
| | speaking anxiety is abnormal and shameful, |
| | ask several friends if they ever felt nervous |
| | before they gave a talk |
| Define Terms | When you label yourself “inferior” or “a |
| | fool” or “a loser”, ask “What is the |
| | definition of ‘a fool’?” You will feel better |
| | when you see that there is no such thing as |
| | “a fool” or “a loser”. |
| The Semantic | Simply substitute language that is less |
| Method | colorful and emotionally loaded. This |
| | method is helpful for “should statements”. |
| | Instead of telling yourself “I shouldn't have |
| | made that mistake”, you can say, “It would |
| | be better if I hadn't made that mistake.” |
| Re-attribution | Instead of automatically assuming that you |
| | are “bad” and blaming yourself entirely for |
| | a problem, think about the many factors |
| | that may have contributed to it. Focus on |
| | solving the problem instead of using up all |
| | your energy blaming yourself and feeling |
| | guilty. |
| Cost-Benefit | List the advantages and disadvantages of a |
| Analysis | feeling (like getting angry when your plane |
| | is late), a negative thought (like “no matter |
| | how hard I try, I always screw up”), or a |
| | behavior pattern (like overeating and lying |
| | around in bed when you're depressed). You |
| | can also use the Cost-Benefit Analysis to |
| | modify a self-defeating belief such as “I |
| | must always try to be perfect”. |

### Crowd-Sourcing the ANT

If the patient chooses not to use machine classification, the patient is provided an option (252) to crowd-source classification. If the patient agrees to crowd-source classification, the server notifies (254) one or more group of members on the platform.

In a preferred embodiment, the patient engagement system (100) chooses a list of members at random to assign the classification request to.

When the patient seeks help for a specific negative thought, each member of the select group of members is notified about the negative thought. If a member is available and chooses (256) to classify the negative thought, the member provides (258) classification input. Further, if the member chooses (260) to also provide a rational response to the negative thought, the member can complete (261) the patient's input by providing the rational response as well. Otherwise, the server can assist the member by identifying (262) an unthinking strategy using a pre-configured logical data structure that allows looking up the right unthinking strategy for a given negative thought and the distortion previously identified. If the member can formulate a rational response after reviewing the unthinking strategy, they can choose (263) to do so. If the member is unable to formulate a rational response, the member's input is marked completed (264) without any rational response for the negative thought.

In a preferred embodiment, the server notifies (266) the patient for each complete input received (complete input includes both classification and rational response) from group members notified by the server. The patient can review (268) the classification provided by a member to either accept or reject the classification.

If the patient accepts the classification received through crowdsourcing, the server can check (270) with the patient if he/she wishes to review the associated rational response as well or provide own rational response. After review (276), the patient can choose (278) to accept or reject the rational response. If the patient accepts the rational response, the server stores (274) the rational response in the user-specific database for future reference. In addition, the corresponding anonymized version of the rational response is stored in the generic database. Further, the negative thought is removed from the list of points to discuss with the therapist if present. Otherwise, the server can provide the patient with an option to send feedback (280) to the member from whom the input was received. The patient can further add (282) the negative thought to the list of points or issues to be discussed with the patient's therapist.

In case the patient opts not to review the rational response after accepting crowdsourced classification, the patient can provide (272) the patient's own rational response for the negative thought. The server stores (274) the rational response provided by the patient in the user-specific database for future reference. In addition, the corresponding anonymized version of the rational response is stored in the generic database. Further, the negative thought is removed from the list of points to discuss with the therapist if present.

The various actions in method 200 may be performed in the order presented, in a different order or simultaneously. Further, in some embodiments, some actions are listed in FIG. 2A to FIG. 2F may be omitted.

### Daily Activation

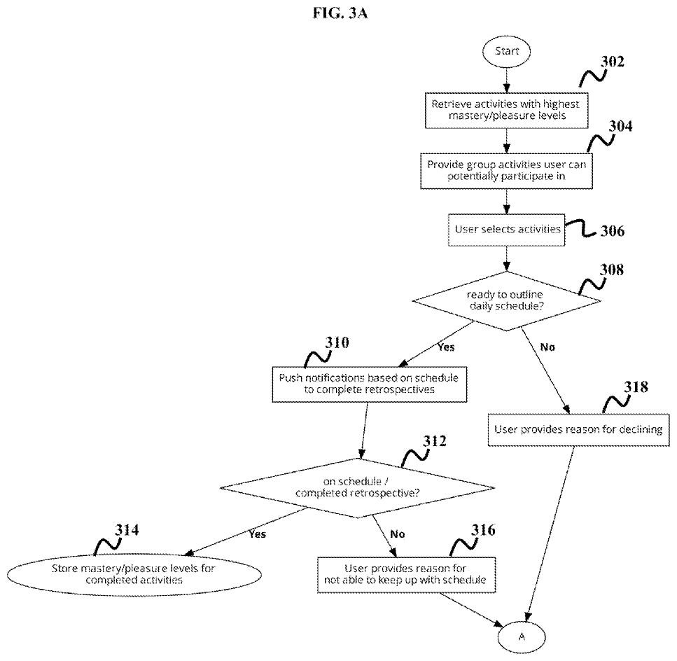

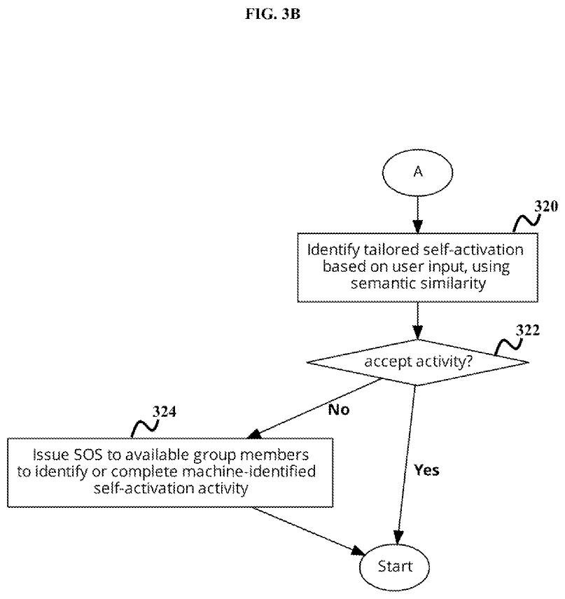

FIG. 3A to FIG. 3B (collectively referred to as FIG. 3) constitute a flow diagram illustrating method for the patient engagement through self-activation activities and daily activity schedule, according to a preferred embodiment.

The patient typically engages with the patient engagement system (100) using one or more activities. When the patient starts interacting with the patient engagement system (100) (for example, using a mobile application), the patient is presented (304) with a list of activities in which the patient can participate. The activities can be activities that involve a group (for example, “The Survey Method”) or individual activities (for example, a daily task such as brushing teeth). The patient can select (306) one or more activities that suit the patient. The list of activities present to the patient can be retrieved (302) from the list of activities with the highest previously recorded mastery/pleasure levels. In a preferred embodiment, steps 302 and 304 happen simultaneously from the patient's perspective.

In a preferred embodiment, the patient engagement system (100) enables the patient to outline a daily schedule involving one or more activities. When the patients choose (308) to outline their schedule, the patient input (318) for the activity schedule is accepted by the server. For the patients that have set up a schedule, the patient engagement system (100) can send (310) notifications (for example, push notifications on mobile on an hourly basis) to help the patients keep up with the schedule. The server regularly checks (312) if the patients are on schedule in completing their activities. When the patients are unable to keep up with their schedule, the patient engagement system (100) can capture (316) reasons for not being able to keep up with the schedule.

The patient can use the information on the patient engagement system (100) (for example, activities completed and any reasons for not being able to complete activities) as a record of activities to engage and discuss with the care provider (for example, psychiatrist or psychotherapist). For the patients that are on schedule, the server notifies them about completing their retrospective for completed activities. As part of the retrospective, the patients are enabled to provide feedback in the form of mastery/pleasure levels for the completed activities. The server stores (314) the mastery/pleasure levels for the user in the user-specific database. In various embodiments, the anonymized mastery/pleasure level data associated with the activities is also stored in the generic database.

In the case where the patient declines to outline a schedule, the patient engagement system (100) can capture (318) the reason for declining the scheduling option.

If the patient is not on schedule or if the patient declines to outline a schedule, the patient engagement system (100) can further analyze the corresponding reason to identify (320) one or more personalized self-activation activities that may be suitable for the patient.

The personalized activities identified can be based on activities stored in the patient engagement system (100) that were chosen by the patients after declining activities. The patient engagement system (100) can identify activities based on the similarity of reason provided before the activities were chosen by the patient. The similarity matching of reasons can be done using the ML module.

The patient can accept or reject the personalized activity suggested by the patient engagement system (100). If the patient rejects the personalized activity chosen by the patient engagement system (100), the patient engagement system (100) can then optionally notify members (via a SOS message) on the patient engagement system (100) to help the patient by identifying a self-activation activity or complete a machine-identified self-activation activity. In various embodiments, the server can enable members on the platform to engage with the patient in a multi-step process and help the patient to identify or complete a self-activation activity.

Table 3 lists some examples of negative thoughts or distortions, and a corresponding self-activation technique that can be used to solve the distortion.

TABLE 3
Examples of distortions and corresponding self-activation
techniques that can be used for the distortion

| Distortion | Self-activation technique |
|---|---|
| Tasks are too difficult and unrewarding | Anti-procrastination Sheet |
| I do not feel like doing anything | Daily record of dysfunctional |
| | thoughts |
| There is no point in doing anything | Pleasure-predicting sheet |
| when I am alone | |
| I am avoiding doing this | But-rebuttal |
| because . . . (excuse) | |
| Whatever I do is not worth much | Self-endorsement |
| I will never be able to complete this task | TIC-TOC Technique |
| There are way too many tasks I must do | Little steps for little feet |
| I should be doing this task instead | Motivation without |
| | coercion |
| I refuse to do anything my mother tells | Disarming technique |
| me to do | |
| I feel like going back to my old ways | Visualizing success |
| I cannot do this on my own without | Count what counts |
| someone telling me to | |
| I just cannot do this | Test you cant's |
| I am afraid of failure | Cannot lose patient |
| | engagement system |

The various actions in method 300 may be performed in the order presented, in a different order or simultaneously.

### Gamification

In a preferred embodiment, the patient engagement system (100) gamifies the patient engagement with the patient engagement system (100) to increase patients' retention rates with CBT and to increase the efficacy of therapeutic benefits. The patients receive points for engaging in various activities. Furthermore, the patients receive points for completing any sort of activity proportional to the qualitative engagement from them, e.g., an ANT automatic classification will not yield as many points as a classification input provided by the patient.

In some embodiments, in addition to gamification using a points patient engagement system (100), the patients may be rewarded with positive thought-evoking animations (for example, confetti) or sounds through the user interface used by the patient to interact with the patient engagement system (100). The patient can pre-configure the animations/sounds that he/she would like to see on completing a set of activities or a schedule. The sounds can be pre-recorded messages either by the patient or another person the patient likes to hear from.

### Advantages

Fogg behavioral model (https://behaviormodel.org/) suggests that three elements must converge at the same moment for a behavior to occur: Motivation, Ability, and Prompt.

The patient engagement system (100) and the various methods described herein reward the patient's behavior using points and/or encouragement, enabling the patients to stay motivated throughout their engagement.

The patient engagement system (100) and the various methods described herein help enhance a patient's ability to navigate through the patient's negative thoughts and engage in the psychotherapy process using various hints and suggestions through automatic classification and crowd-sourcing inputs.

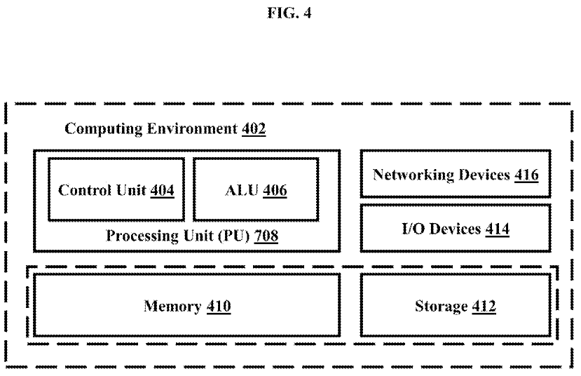

FIG. 4 is a block diagram illustrating the computing environment for an application server enabling the methods disclosed herein, according to an embodiment.

As depicted the computing environment 402 comprises at least one processing unit 708 that is equipped with a control unit 404 and an Arithmetic Logic Unit (ALU) 406, a memory 410, a storage unit 412, a plurality of network devices 416 and a plurality Input-output (I/O) devices 414. Processing unit 408 is responsible for processing the instructions of the schemes. The processing unit 408 receives commands from the control unit to perform its processing. Further, any logical and arithmetic operations involved in the execution of the instructions are computed with the help of ALU 406.

The overall computing environment 402 can be composed of multiple homogeneous and/or heterogeneous cores, multiple CPUs of various kinds, special media, and other accelerators. Processing unit 408 is responsible for processing the instructions of the scheme. Further, the plurality of processing units 408 may be located on a single chip or over multiple chips.

The algorithm comprises of instructions and codes required for the implementation are stored in either the memory unit 410 or the storage 412 or both. At the time of execution, the instructions may be fetched from the corresponding memory 410 and/or storage 412 and executed by the processing unit 408.

With the increase in the number of connected devices, the information security risk associated with the patient engagement system (100) also increases.

Various networking devices or external I/O devices may be connected to the computing environment to support the implementation through networking interfaces 616 and the I/O interfaces 414.

The embodiments disclosed herein can be implemented through at least one software program running on at least one hardware device and performing network management functions to control the network elements. The network elements shown in FIG. 1 and FIG. 4 include blocks which can be at least one of a hardware device, or a combination of hardware device and software module.

The embodiments disclosed herein specify the patient engagement system (100) for increasing patient engagement in psychotherapy. Therefore, it is understood that the scope of the protection is extended to such a program and in addition to a computer-readable means having a message therein, such computer-readable storage means contain program code means for implementation of one or more steps of the method when the program runs on a server or mobile device or any suitable programmable device. The method is implemented in a preferred embodiment through or together with a software program written in e.g., Very high-speed integrated circuit Hardware Description Language (VHDL) another programming language or implemented by one or more VHDL or several software modules being executed on at least one hardware device.

The hardware device can be any kind of device which can be programmed including e.g., any kind of computer like a server or a personal computer, or the like, or any combination thereof, e.g., one processor and two FPGAs. The device may also include means which could be e.g., hardware means like e.g., an ASIC, or a combination of hardware and software means, e.g., an ASIC and an FPGA, or at least one microprocessor and at least one memory with software modules located therein. Thus, the means are at least one hardware means and/or at least one software means. The method embodiments described herein could be implemented in pure hardware or partly in hardware and partly in software. The device may also include only software means. Alternatively, the invention may be implemented on different hardware devices, e.g., using a plurality of CPUs.

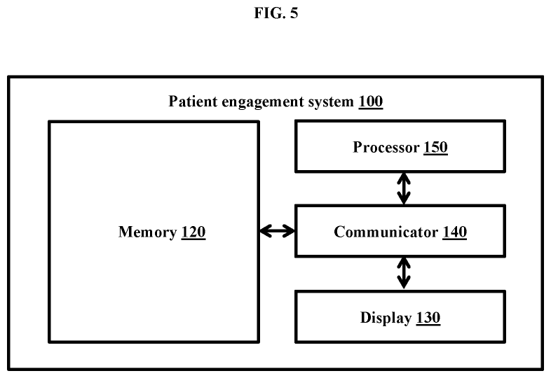

FIG. 5 is a block diagram of the patient engagement system (100) for the patient engagement, according to another embodiment herein. The patient engagement system (100) helps to facilitate cognitive behavioral therapy by helping patients complete the triple-column technique. A patient who has a negative thought is asked to cite a cognitive distortion for the said thought and create a rational response or positive twist on that negative thought. The cognitive distortion can be identified by the patient, the patient engagement system (100) using the ML model (116), or the patient's team members. The rational response can be created by the patient, retrieved if previously stored by the patient engagement system (100), or crowd-sourced from a teammate (i.e., second patient). Teams consisting of three or more patients compete against each other and are ranked using a point system. The points are awarded to each patient proportional to their engagement with the app.

Examples of the patient engagement system (100) include, but are not limited to a smartphone, a kiosk, a tablet computer, a Personal Digital Assistance (PDA), a desktop computer, an Internet of Things (IoT), a wearable device, etc. In another embodiment, the patient engagement system (100) includes a processor (150), a memory (120), a display (130), and a communicator (140), where the display (130) is a physical hardware component that can be used to display a content/screen (e.g. home screen) to the user of the patient engagement system (100). Examples of the display (130) include, but are not limited to, a light-emitting diode display, a liquid crystal display, etc. The processor (150) is implemented by processing circuitry such as logic gates, integrated circuits, microprocessors, microcontrollers, memory circuits, passive electronic components, active electronic components, optical components, hardwired circuits, or the like, and may optionally be driven by a firmware. The circuits may, for example, be embodied in one or more semiconductor chips, or on substrate supports such as printed circuit boards and the like. The processor (150) may be a general-purpose processor, such as a Central Processing Unit (CPU), an Application Processor (AP), or the like, a graphics-only processing unit such as a Graphics Processing Unit (GPU), a Visual Processing Unit (VPU) and the like. The processor (150) may include multiple cores to execute the instructions.

The processor (150) is configured to execute instructions stored in the memory (120). The applications include the ML model (116) installed in the patient engagement system (100) are stored in the memory (120). The memory (120) comprises the databases (108, 110, 104, 106). The memory (120) stores instructions to be executed by the processor (150). The memory (120) may include non-volatile storage elements. Examples of such non-volatile storage elements may include magnetic hard discs, optical discs, floppy discs, flash memories, or forms of electrically programmable memories (EPROM) or electrically erasable and programmable (EEPROM) memories. In addition, the memory (120) may, in some examples, be considered a non-transitory storage medium. The term “non-transitory” may indicate that the storage medium is not embodied in a carrier wave or a propagated signal. However, the term “non-transitory” should not be interpreted that the memory (120) is non-movable. In some examples, the memory (120) can be configured to store larger amounts of information than its storage space. In certain examples, a non-transitory storage medium may store data that can, over time, change (e.g., in Random Access Memory (RAM) or cache). The memory (120) can be an internal storage unit, or it can be an external storage unit of the patient engagement system (100), a cloud storage, or any other type of external storage.

The processor (150) receives an input from a first patient from a plurality of patients, where the input indicates that the first patient has an Automatic Negative Thought (ANT). Further, the processor (150) displays a first option to participate in a triple-column associated with the ANT of the first patient, a second option for assistance of at least one second patient of the plurality of patients, and a third option for assistance of an application. Further, the processor (150) detects the first option, or the second option or the third option selected by the first patient.

When the first option is selected by the first patient, then the processor (150) displays a triple-column (i.e., a triple-column table). Further, the processor (150) receives an input corresponding to the ANT, a cognitive distortion and a rational response in the triple-column table from the first patient. Further, the processor (150) allocates a score to the first patient based on the input provided by the first patient.

When the second option is selected by the first patient, then the processor (150) sends a cognitive distortion and rational response request to second patients (i.e., teammates/group-members of the first patient) in the plurality of patients. Further, the processor (150) receives the cognitive distortion and the rational response for the ANT from the second patient. Further, the processor (150) allocates the score to a second patient and the first patient upon acceptance of the first patient for the cognitive distortion and the rational response for the ANT received from that second patient.

When the third option is selected by the first patient, the processor (150) predicts the cognitive distortion and the rational response for the ANT using the ML model (116) associated with the application. Further, the processor (150) allocates the score to the first patient upon acceptance of the first patient for the cognitive distortion and the rational response for the ANT predicted by the ML model (116).

The processor (150) stores the cognitive distortion and the rational response for the ANT into the memory (120).

In an embodiment, the processor (150) adds the score to a previously allocated score of that second patient, and the first patient upon acceptance of the first patient for the cognitive distortion and the rational response for the ANT of the first patient received from that second patient.

In another embodiment, the processor (150) adds the score to the previously allocated score of the first patient upon acceptance of the first patient for the cognitive distortion and the rational response predicted by the ML model (116) for the ANT of the first patient.

In another embodiment, the processor (150) adds the score to a previously allocated score of the first patient upon filling the triple-column table by the first patient.

In an embodiment, the processor (150) detects a rejection of the first patient on the cognitive distortion and the rational response for the ANT received from the ML model (116) or the second patient. Further, the processor (150) stores the ANT to the problematic thoughts database (110). Further, the processor (150) displays the ANT in the problematic thoughts database (110) to the therapist during the in-person group therapy session or based on user input. Further, the processor (150) receives the cognitive distortion and the rational response for the ANT from the therapist and displays the cognitive distortion and the rational response received from the therapist to the first patient.

In an embodiment, the processor (150) determines whether a crowd stipend of the first patient meets a crowd stipend threshold. Further, the processor (150) sends the cognitive distortion and rational response request to the second patient when the crowd stipend of the first patient meets the crowd stipend threshold. Further, the processor (150) decrements the crowd stipend of the first patient.

In an embodiment, the processor (150) receives the cognitive distortion for the ANT and a rationale for selecting the cognitive distortion from the second patients. Further, the processor (150) displays the untwist-your-thinking strategy activity to be performed by the second patients based on the cognitive distortion when only the cognitive distortion for the ANT and a rationale for selecting the cognitive distortion is received from the second patients. Further, the processor (150) receives the rational response for the ANT upon performing the untwist-your-thinking strategy by the second patient.

In an embodiment, the processor (150) sends the cognitive distortion and the rational response for the ANT to the first patient. Further, the processor (150) receives an input indicating acceptance of the cognitive distortion and rejection of the rational response by the first patient. Further, the processor (150) displays an untwist-your-thinking strategy activity to be performed by the first patient based on the input and the cognitive distortion. Further, the processor (150) receives the rational response for the ANT upon performing the untwist-your-thinking strategy by the first patient.

In an embodiment, the processor (150) receives the score allocated to each patient of the plurality of patients, where the plurality of patients is associated with a group/team. Further, the processor (150) assigns a score to the group by combining the score of each patient of the plurality of patients associated with the group. Further, the stores the score of the group and the score allocated to each patient of the plurality of patients into the memory (120). Further, the processor (150) displays the score and a rank of each group on the display (130)

In an embodiment, the processor (150) retrieves a previously stored ANT similar to the ANT using the ML model (116), and a cognitive distortion and a rational response of the previously stored ANT from the memory (120) for prediction. In another embodiment, the processor (150) assigns a cognitive distortion for an unrecognized ANT. In an embodiment, the processor (150) allocates a score to the first patient and the group proportional to an engagement of the first patient with the patient engagement system (100), and the therapist of the first patient.

The communicator (140) is configured for communicating internally between hardware components in the patient engagement system (100). Further, the communicator (140) is configured to facilitate communication between the patient engagement system (100) and other devices via one or more networks (e.g., Radio technology). The communicator (140) includes an electronic circuit specific to a standard that enables wired or wireless communication.

A function associated with the ML model (116) may be performed through the non-volatile/volatile memory (120), and the processor (150). The one or a plurality of processors (150) control the processing of the input data in accordance with a predefined operating rule or the ML model (116) stored in the non-volatile/volatile memory (120). The ML model (116) may consist of a plurality of neural network layers. Each layer has a plurality of weight values and performs a layer operation through the calculation of a previous layer and an operation of a plurality of weights. Examples of neural networks include, but are not limited to, convolutional neural network (CNN), deep neural network (DNN), recurrent neural network (RNN), restricted Boltzmann Machine (RBM), deep belief network (DBN), bidirectional recurrent deep neural network (BRDNN), generative adversarial networks (GAN), and deep Q-networks.

Although FIG. 5 shows the hardware components of the patient engagement system (100), but it is to be understood that other embodiments are not limited thereon. In other embodiments, the patient engagement system (100) may include less or a greater number of components. Further, the labels or names of the components are used only for illustrative purposes and do not limit the scope of the invention. One or more components can be combined to perform the same or substantially similar function for patient engagement.

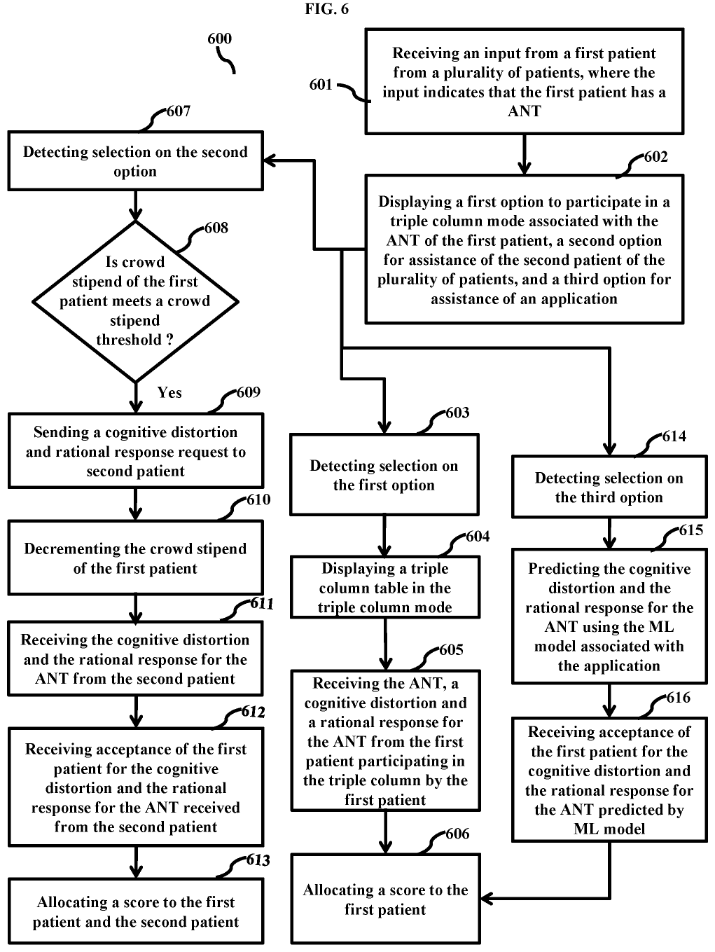

FIG. 6 is a flow chart (600) illustrating a patient engagement method, according to embodiments as disclosed herein. In an embodiment, the patient engagement method allows the processor (150) to perform steps 601 to 615 of the flow chart (600). At 601, the method includes receiving the input from the first patient from the plurality of patients, where the input indicates that the first patient has the ANT. At 602, the method includes displaying the first option to participate in the triple-column associated with the first ANT of the first patient, the second option for assistance of the second patient of the plurality of patients, and the third option for assistance of the application. At 603 and 604, upon detecting the selection on the first option, the method includes displaying the triple-column table. At 605, the method includes receiving the first ANT, the cognitive distortion and the rational response for the first ANT from the first patient participating in the triple-column by the first patient. At 606, the method includes allocating the score to the first patient.

At 607 and 608, upon detecting the selection of the second option, the method includes determining whether the crowd stipend of the first patient meets the crowd stipend threshold. At 609, the method includes sending the cognitive distortion and the rational response request to the second patient when the crowd stipend of the first patient meets the crowd stipend threshold. At 610, the method includes decrementing the crowd stipend of the first patient. At 611, the method includes receiving cognitive distortion and the rational response for the ANT from the second patient. At 612, the method includes receiving acceptance of the first patient for the cognitive distortion and the rational response for the ANT received from the second patient. At 613, the method includes allocating the score to the first patient and the second patient.

At 614 and 615, upon detecting selection on the third option, the method includes predicting the cognitive distortion and the rational response for the ANT using the ML model (116) associated with the application. At 616, the method includes receiving acceptance of the first patient for the cognitive distortion and the rational response for the ANT predicted by the ML model (116) and continues step 606.

The various actions, acts, blocks, steps, or the like in the flow diagram (600) may be performed in the order presented, in a different order, or simultaneously. Further, in some embodiments, some of the actions, acts, blocks, steps, or the like may be omitted, added, modified, skipped, or the like without departing from the scope of the invention.

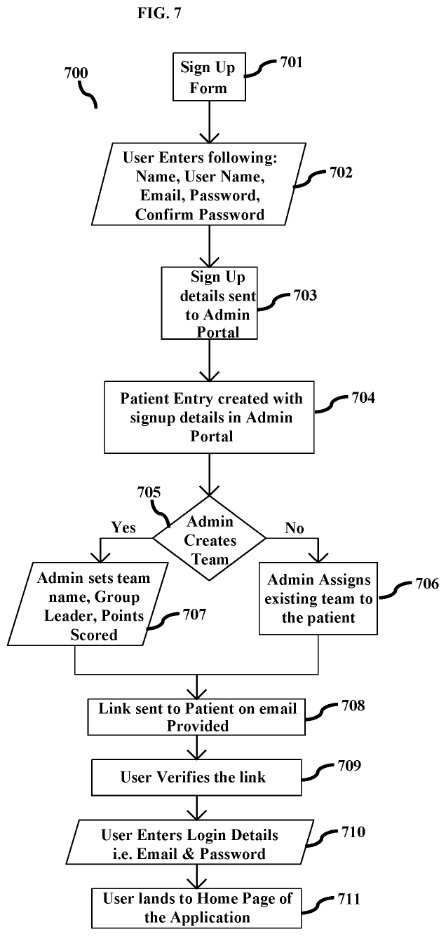

FIG. 7 is a flow chart (700) illustrating a method for signing up the patient to the patient engagement system (100), according to embodiments as disclosed herein. The flow chart (700) includes steps 701 to 711. The patient engagement system (100) allows the patients to register for the usage of the patient engagement system (100) and log in to the application upon approval by an admin of the patient engagement system (100). The patient engagement system (100) assigns the crowd stipend to the patient and tallies an individual point scored by the patient. The patient engagement system (100) allows the patient to see a team leaderboard of the patient including information of team ranks among other teams, and the point of the team (also called as group) of the patient scored. The patient engagement system (100) allows the patient to score team points and individual points by resolving the patient's own ANT.

The patient engagement system (100) allows the patient to earn team points and individual points by resolving the ANT of any other patient from the patient's team. The patient engagement system (100) allows the patient to earn the team points and individual points if the patient formulates the rational response and the patient's ANT is resolved by the application. The patient engagement system (100) allows the patient to earn team points and individual points by requesting the patient's team members to resolve the ANT of the patient. The patient engagement system (100) allows the patient to see teammates' responses of the patient against the patient's crowd-sourced ANT, the patient engagement system's responses for a system-queried ANT and to like or dislike the response, where the second patient and the first patient are teammates. The patient engagement system (100) allows the patient to log out from the application.

The patient engagement system (100) allows the admin to receive and approve/disapprove signup requests of the patient in the application. The patient engagement system (100) allows the admin to create a new team and assign the team to the patient. The patient engagement system (100) allows the admin to assign an existing team to the patient. The patient engagement system (100) allows the admin to view all teams and the team members along with the teams' scores. The patient engagement system (100) allows the admin to view all the patient's records and their scores.

At 701, when the patient lands on a signup page of the patient engagement system (100), then at 702 the patient (i.e., patient) can provide the following information to the patient engagement system (100) to log in:
Name: An input greater than 20 characters (first and last name of the patient),
Email: Input field that is a valid email of the patient,
Username: An input greater than 20 characters,
Password: A protected input with at least 8 characters including one numeric digit, one character & one uppercase and lowercase alphabetical character,
Confirm Password: This field should match the “Password” field input. In case of a mismatch, the patient engagement system (100) displays a message saying “The passwords don't match. Please try again.”

At 703, once the user fills all of these fields of the patient engagement system (100), then the user can click on the signup button of the application. At 704, the application validates all the fields and if everything is according to the listed validations above, a record of this signup request will get generated in the admin portal of the application in a ‘Signup Requests’ section. If the inputs do not match the validations mentioned above, then the patient engagement system (100) displays an error message to the patient saying ‘Please provide the correct input to proceed’ below the invalid field.

At 705, once a signup request is generated, the patient engagement system (100) sends the request to the admin portal where the admin will decide whether to approve or reject this signup request. If the admin approves the received request, then the patient engagement system (100) creates a patient record in the admin portal with the user-provided information. If the admin disapproves or rejects the signup request, then the patient engagement system (100) removes the request from the admin portal and sends a notification to the patient that the request has been disapproved or rejected. Once a patient entry is created, the patient engagement system (100) allows the admin to assign one team to the patient. The patient engagement system (100) follows the rules listed below for assigning the team to the patient:
1. The admin will be able to assign multiple patients to a single team at a single time.
2. At 705, the admin will be able to create a new team and can assign it to the patients who aren't part of any team yet.
3. At 706, the admin will be able to assign an already-created team to multiple unassigned patients at a time.
4. At 707, a team will have the following attributes:
   a. Team Name
   b. Group Leader
   c. Points scored
5. A patient will have the following attributes:
   a. Name
   b. Email
   c. Username
   d. Password
   e. Points Scored
   f. Group Leader Status (T/F)
   g. Team Name
   h. Crowd Stipend

At 708, once the team is assigned to the patient, the patient engagement system (100) sends a confirmation email along with a verification link to the patient's provided email address. At 709-711, when the patient clicks on that link, then the patient engagement system (100) opens the application with a login screen where the patient will be able to log in and proceed to a home screen. If the patient provides invalid credentials while logging in, then the patient engagement system (100) displays an ‘Invalid Credentials’ message on the display (130).

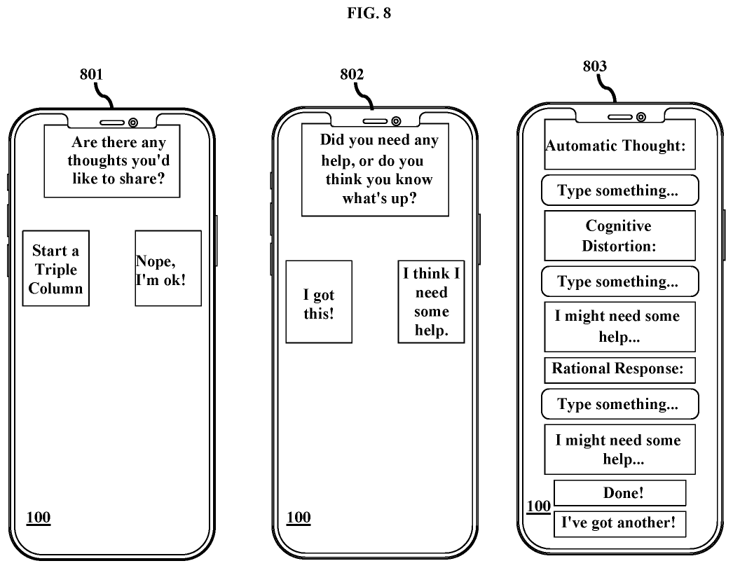

FIG. 8 illustrates an example scenario of providing the triple-column to the patient by the patient engagement system (100), according to embodiments as disclosed herein. As shown in 801 of the FIG. 8, whenever the first patient lands on the home screen of the application installed in the patient engagement system (100), the patient engagement system (100) allows the first patient to click on a button ‘ANT’ i.e., ‘Automatic Negative Thought’. When the first patient clicks on this button, the patient engagement system (100) enquires to the first patient for any negative thought. If the first patient replies by clicking on ‘Nope, I am ok’, then the patient engagement system (100) directs the first patient to the home screen. If the first patient clicks on the ‘Start a Triple-column’, then the patient engagement system (100) directs the first patient to another screen as shown in 802 of FIG. 8. If the first patient knows about the cognitive distortion and its rational response of the ANT, the patient engagement system (100) allows the first patient to click on the ‘I got this’ option and further the user will be directed to the screen shown in 803 of the FIG. 8. Upon filling each input field: ANT, the cognitive distortion, the rational response shown in 803 of FIG. 8 by the first patient, the patient engagement system (100) allocates 5 points to the first patient.

Consider an example scenario, in which the first patient having some negative thoughts and for clarity and closure, the first patient starts the application (i.e. patient engagement system (100)), clicks on the “ANT” button and fills in the negative thought of the first patient and then proceeds to view the screen shown in 803 of the FIG. 8 after clicking “I got this” option. Now the first patient also recognizes the negative thought type and classifies the cognitive distortion for the ANT as well. Now the first patient also fills in the rational response against the ANT and clicks on ‘Done’. This whole scenario will lead to an increment of 15 points in the current score of the first patient, and the first patient will now see an option to land on the home screen where the patient can enter another triple-column entry as well. These 15 points will be allotted to the first patient's respective team and leaderboard stats will change accordingly.

The first patient will only be able to add and edit the rational response field if the first patient has already filled the cognitive distortion field. If the first patient has not provided input against all fields, the first patient will not be able to click either of the ‘Done’, ‘I've Got Another’ buttons. The first patient will not be able to click and proceed to the ‘I think I might need some help’ button under the rational response if the first patient has not provided the cognitive distortion.

Whenever the first patient will click on the “I might need some help option” in the screen shown in 803 of the FIG. 8, after providing ANT but needs help with the cognitive distortion or the rational response, the first patient will be directed to the following options by the patient engagement system (100):
‘Ask A Friend’:—If the first patient clicks on this option, it implies the first patient would like to ask help from the teammates and will be directed to the respective screen.
‘Maybe you could help me’:—This option implies asking for help from the patient engagement system (100). The first patient will be directed to this option's respective screen.

When the first patient clicks on “I've Got another” button, then the patient engagement system (100) directs the first patient to the same screen of ‘Start A triple-column’ following the flow mentioned in that respective section.

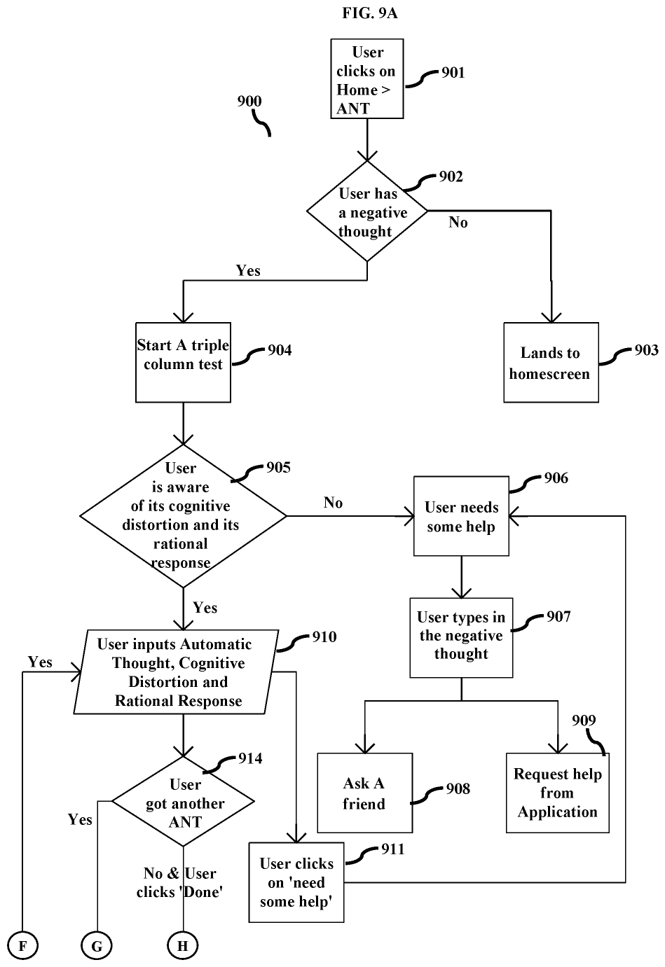

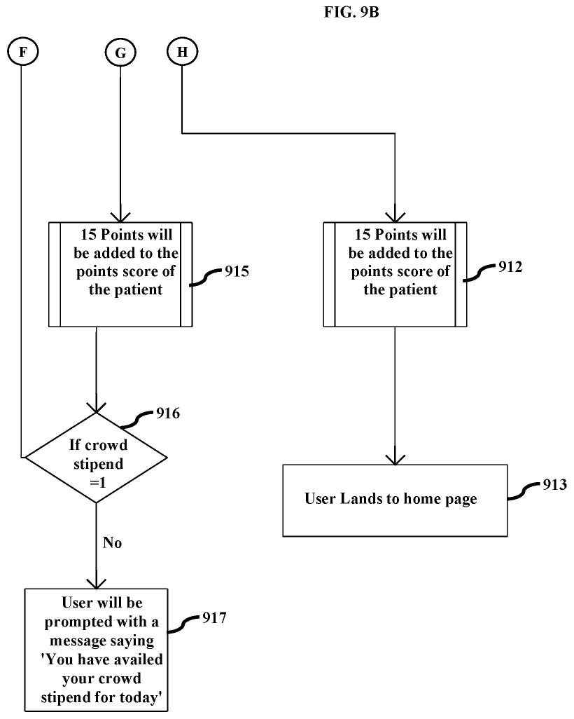

FIG. 9A to FIG. 9B (collectively referred to as FIG. 9) constitutes a flow chart (900) illustrating an overall flow of the patient engagement method, according to embodiments as disclosed herein. The flow chart (900) includes the steps 901-917. At 901, the first patient reaches the home screen of the patient engagement system (100), in which the first patient can give input on the ANT option displayed on the home screen when the first patient has the ANT. At 902 and 903, when the first patient doesn't have the ANT, the first patient stays on the home screen. At 902 and 904, upon inputting on the ANT option displayed in the home screen by the first patient, the patient engagement system (100) detects the ANT of the first patient and starts the triple-column with the first patient. At 905 and 906, when the first patient is not aware of the cognitive distortion and the rational response of the ANT, then the first patient needs the help of other entities. At 907, when the first patient needs the help of other entities, then the first patient inputs the ANT to the patient engagement system (100), and further the first patient can choose either the assistance of the friend (i.e., second patient) at 908 or the application at 909.

At 905 and 910, when the first patient is aware of the cognitive distortion and the rational response of the ANT, then the first patient inputs the ANT, the cognitive distortion and the rational response to the patient engagement system (100). At 911, even after providing the ANT, the cognitive distortion and the rational response to the patient engagement system (100), the first patient can ask for help of the application of the patient engagement system (100), and further, the patient engagement system (100) continues to step 906. At 914, 912 and 913, when the first patient hasn't another ANT to resolve, then the patient engagement system (100) adds 15 points to the score of the first patient and directs the first patient to the home screen. At 914, 915 and 916, when the first patient has another ANT to resolve, then the patient engagement system (100) adds 15 points to the score of the first patient and checks whether the first patient has the minimum crowd stipend (i.e., 1) to request the assistance of the teammate. If the first patient doesn't have the minimum crowd stipend (i.e. 1) to request the assistance of the teammate, then the patient engagement system (100) displays the first patient with a message saying ‘You have availed your crowd stipend for today’. If the first patient has the minimum crowd stipend (i.e., 1) to request the assistance of the teammate, then the patient engagement system (100) continues to step 910.

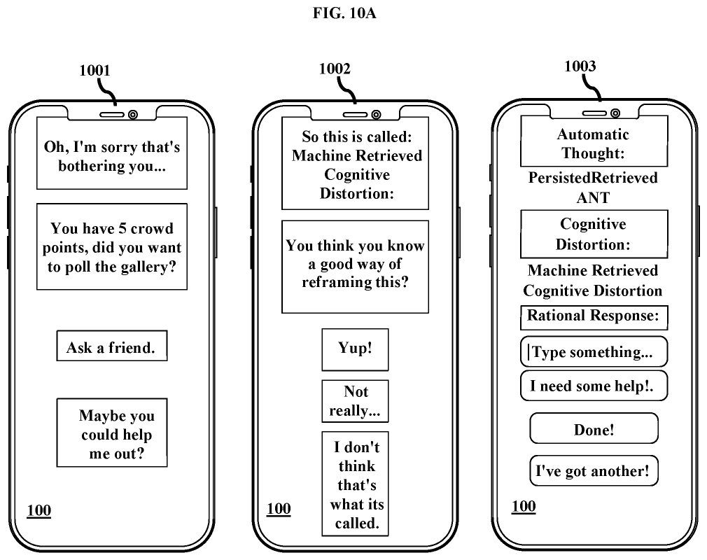

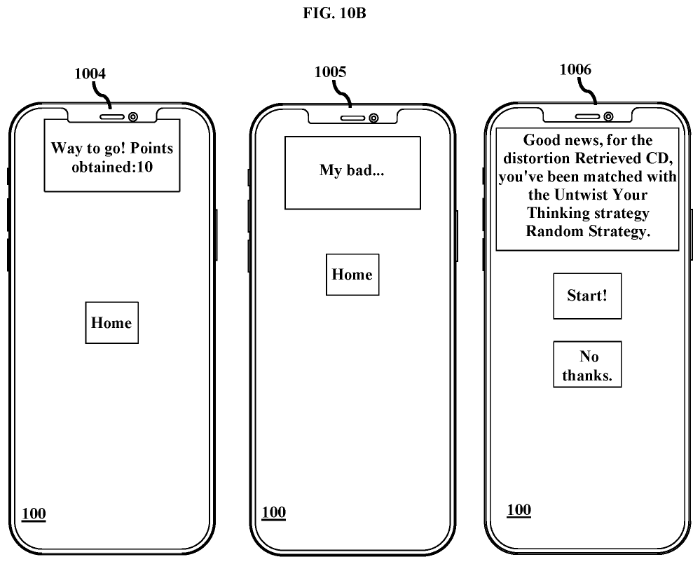

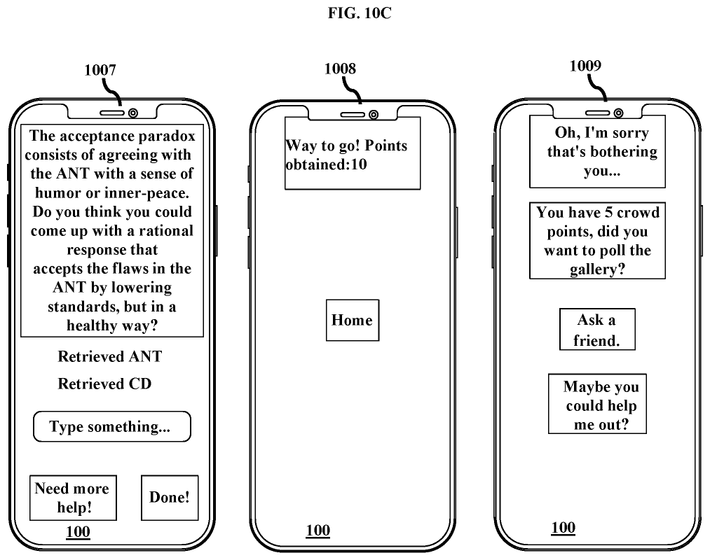

FIG. 10A to FIG. 10C (collectively referred to as FIG. 10) illustrate an example scenario of assisting with the application or the teammate (i.e., second patient) to the first patient for providing the cognitive distortion the rational response of the ANT of the patient, according to embodiments as disclosed herein. As shown in 1001 of the FIG. 10, whenever the first patient suffers from any negative thought and is not able to resolve and identify it (classify it with a cognitive distortion), the first patient will be presented with two options in the patient engagement system (100) i.e., either ask for help from a friend (teammate) or ask for help from the application (the first patient can ask for help from the application by clicking on the option “Maybe you could help me out?”.

As shown in 1002 of the FIG. 10, when the first patient asks for help from the application by clicking on “Maybe you could help me out?” option, then the patient engagement system (100) first invokes its semantically similar behavior mechanism and checks whether a semantically similar ‘ANT’ with the same username of the first patient exists in the database or not. If the similar data is not retrieved, then the patient engagement system (100) displays a probable cognitive distortion only based on its ANT. If similar data is retrieved, then the patient engagement system (100) displays cognitive distortion and the rational response if requested.

As shown in 1003 of FIG. 10, the first patient has the liberty to agree or disagree with the cognitive distortion predicted by the patient engagement system (100). If the first patient disagrees with the predicted cognitive distortion, then the patient engagement system (100) sets the ANT as a problematic ANT and stores it in the problematic ANT table. If the first patient agrees to the predicted cognitive distortion, then the first patient can click on ‘Yup’ on the screen of the patient engagement system (100). Further, the first patient is allowed to provide a rational response in the next step. Once the first patient provides the rational response to the patient engagement system (100) and clicks on ‘Done’, then the patient engagement system (100) performs the steps listed below:
Store the resolved ANT with the fields ‘Username, ANT, Cognitive Distortion and Rational Response’ in the triple-column table.
Allocate 10 points to the first patient and 10 points to the team of the first patient.

Whenever the first patient generates the request to ask for help from the application, this ANT record will move to the problematic ANTs table. When the ANT is resolved, the record of the ANT will get removed from the problematic ANT table and a group request table if the ANT exists in these tables. The first patient will see the screen as shown in 1004 of FIG. 10, after clicking on ‘Done’ where the first patient will see that the first patient has been awarded 10 points. Now the first patient can either go to the home screen or will be able to generate a new ANT request by clicking on ‘I've got another’ (not shown).

If the first patient agrees to the predicted cognitive distortion but doesn't know the rational response against the cognitive distortion’ and asks the patient engagement system (100) to provide the rational response as well by clicking on ‘Not Really’, if the patient engagement system (100) has retrieved a semantically similar entry that matched the username of the first patient, this rational response will be retrieved from the database. If a semantically similar entry does not exist or if the first patient doesn't agree to this response, then the patient engagement system (100) will match the cognitive distortion with the untwist your strategy thinking activity. Further, the patient engagement system (100) will ask the first patient to perform the activity if the first patient wants. If the first patient does agree to the rational response generated by the patient engagement system (100) (accepts the system-retrieved rational response), then the patient engagement system (100) performs the steps listed below:
Allocate 5 points to the first patient.
Allocate 5 points to the team of the first patient.

As mentioned, whenever the first patient generates a request to ask for help from the application, this ANT record will move to the problematic ANTs table. Now the first patient can either go to the home screen or will be able to generate the new ANT request by clicking on ‘I've got another’. If the first patient doesn't agree to the cognitive distortion predicted by the patient engagement system (100) and clicks on ‘I don't think this is what it is called’ or does not participate in the untwist-your-thinking strategy activity, the first patient will be taken to the screen shown in 1005 of the FIG. 10 from where the first patient will be able to go to the home screen. In this case, the ANT will remain on the problematic ANT table or the group request table (if applicable).

Whenever the first patient asks for help from the application and the patient engagement system (100) couldn't find semantically similar behavior and the first patient accepts the cognitive distortion, then the first patient needs to enter the rational response. If the first patient cannot, then the first patient will be redirected to perform the untwist-your-thinking strategy activity. As shown in 1006 of FIG. 10, the first patient will see only an example as many strategies could be presented. Based on the cognitive distortion, the patient engagement system (100) suggests a type of strategy using a dictionary maintained in a backend. The cognitive distortions and their respective strategy types are given in table 4.

TABLE 4
“All-or-Nothing Thinking”: [“Acceptance Paradox”, “Be Specific”,
“Semantic Method”],
“Overgeneralization”: [“Acceptance Paradox”, “Be Specific”,
“Let's Define Terms”]
“Mental Filter”: [“Acceptance Paradox”, “Be Specific”, “Reattribution”,
“Semantic Method”]
“Disqualifying the Positive”: [“Acceptance Paradox”, “Be Specific”]
“Mind Reading”: [“Acceptance Paradox”, “Be Specific”, “Paradoxical
Magnification”, “Let's Define Terms”]
“Fortune Teller Error”: [“Acceptance Paradox”, “Be Specific”,
“Paradoxical Magnification”, “Let's Define Terms”]
“Magnification”: [“Acceptance Paradox”, “Paradoxical Magnification”,
“Semantic Method”]
“Minimization”: [“Acceptance Paradox”, “Paradoxical Magnification”,
“Semantic Method”]
“Emotional Reasoning”: [“Acceptance Paradox”, “Be Specific”,
“Paradoxical Magnification”, “Let's Define Terms”, “Semantic Method”]
“Should Statement”: [“Acceptance Paradox”, “Reattribution”,
“Semantic Method”]
“Labeling”: [“Acceptance Paradox”, “Be Specific”, “Reattribution”,
“Paradoxical Magnification”, “Semantic Method”]
“Personalization”: [“Acceptance Paradox”, “Be Specific”, “Reattribution”,
“Paradoxical Magnification”, “Let's Define Terms”, “Semantic Method”]

If the first patient clicks on ‘No Thanks’, then the first patient-generated ANT along with the username and the cognitive distortion will be listed in the problematic ANT table and now the first patient will be asked if the first patient has another ‘ANT’ or not. If the first patient clicks on ‘I've got another’, then the first patient will be taken to the respective screen. If the first patient has no other ‘ANT’, then the first patient will be taken to the home screen of the patient engagement system (100).

To participate in the untwist-your-thinking strategy activity, the first patient clicks on the ‘Start’ button on the screen shown in 1006. Once the first patient clicks on this option, the first patient will be matched to a different untwist-your-thinking strategy according to the recorded cognitive distortion. For example, the screen shown in 1007 of FIG. 10 is the Acceptance Paradox activity.

On the screen shown in 1007, the first patient is urged to add the rational response against the first patient's entered ANT and the application retrieved cognitive distortion. Here if the first patient adds the rational response and clicks on ‘Done’, then the patient engagement system (100) performs the steps listed below:
Store the resolved ANT along with details ‘Username, ANT, Cognitive Distortion and Rational Response’ in the ‘Triple-column table’.
Allocate 10 points to the first patient and 10 points to the team.

As mentioned, whenever the first patient generates the request to ask for help from the application, this ANT record will move to the problematic ANTs table. When the ANT is resolved, this ANT will get removed from the problematic ANT table and the group request table if it exists in these tables.

The first patient will only be able to click on ‘Done’ if the first patient has input both the cognitive distortion and the rational response. If the first patient has not added the rational response and clicks on ‘Done’, the application will display a message saying, ‘Please add your Rational Response in order to continue’.

The first patient will see the screen shown in 1008 of FIG. 10 after clicking on ‘Done’ where the first patient will see that the first patient has been awarded 10 points. Now the first patient can either go on the home screen or will be able to generate the new ANT request by clicking on ‘I've got another’ (not shown).

Whenever the first patient suffers from the negative thought and is not able to resolve and/or identify it, the first patient will be displayed with two options i.e., either ask for help from the friend or ask for help from the application shown in 1009 of the FIG. 10. If the first patient clicks on the ‘Ask a Friend’ option on the screen shown in 1009 of FIG. 10, the patient engagement system (100) will check whether the first patient's crowd stipend is greater than or equal to one or not. This means the first patient will only be able to take help from the friend if the first patient's crowd stipend is greater than 0.

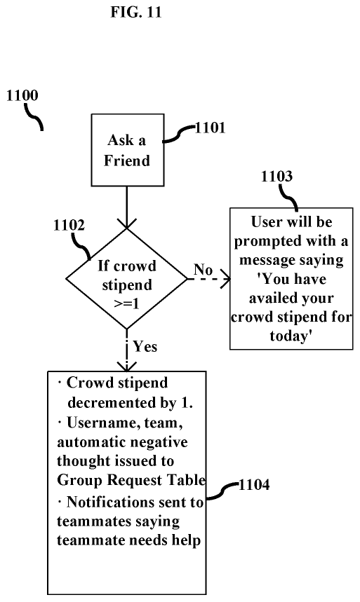

FIG. 11 is a flow chart (1100) illustrating a method of consuming the crowd stipend, according to embodiments as disclosed herein. The flow chart (1100) includes the steps 1101-1104. At 1101, the first patient clicks on the ‘Ask a Friend’ option on the screen of the patient engagement system (100) as shown in 1009 of FIG. 10. At 1102, the patient engagement system (100) checks whether the first patient's crowd stipend is greater than or equal to one or not. This means the first patient will only be able to take help from the friend if the first patient's crowd stipend is greater than zero. At 1103, when the crowd stipend is zero, then the patient engagement system (100) prompts the first patient with a message saying, ‘You have availed your crowd stipend for today’. At 1104, when the crowd stipend is greater than zero, then the patient engagement system (100) decrements the crowd stipend by 1. Further, the patient engagement system (100) saves the username, the team, the ANT issued to the group request table, and sends notifications to the teammates saying the first patient needs help.

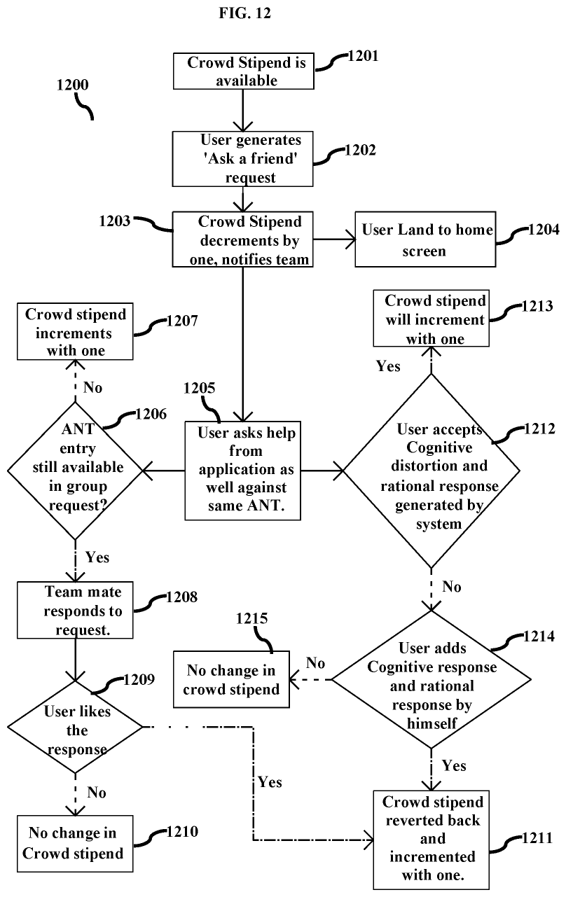

FIG. 12 is a flow chart (1200) illustrating a method of handling the crowd stipend and providing cognitive distortion and the rational response to the first patient, according to embodiments as disclosed herein. The flow chart (1200) includes the steps 1201-1215. The patient engagement system (100) allocates the crowd stipend to each patient every week, where the crowd stipend is a score. Every week, each patient's crowd stipend will get replenished to 5 automatically by the patient engagement system (100). So, every patient will be able to avail ‘Ask a Friend’ option 5 times a week against the requested ‘ANTs’. So, utilizing the ‘Ask a Friend’ option will decrement the patient's crowd stipend by 1 point. If the patient has no crowd stipend left and clicks on the ‘Ask a Friend’ option, then the patient engagement system (100) displays the following message: ‘Unfortunately, you have availed your crowd stipend for this week!’ to the patient.

At 1201-1203, if the first patient has the crowd stipend available and clicks on the ‘Ask a friend’ option, then the patient engagement system (100) decrements the crowd stipend of the first patient by 1 and generates a group request for the first patient's group by inputting an entry into the group request table with the username, the team name, and the ANT attached to that entry. Further, the patient engagement system (100) sends the notification to the group members (i.e., second patients, teammates, etc.) displaying a message ‘Your team member has requested some help with a tricky ANT!’. At 1204, the patient engagement system (100) directs the first patient to the home screen. Once the request to ‘Ask for a friend’ is generated but meanwhile, the first patient takes help from the application at 1205, the first patient accepts the cognitive distortion and the rational response generated by the patient engagement system (100) at 1212, then the patient engagement system (100) increments the crowd stipend by one at 1213, as the group request entry will get removed from the table (and therefore revoked).

At 1214 and 1211, once the request to ‘Ask for a friend’ is generated but meanwhile the first patient resolves the cognitive response and the rational response, then the patient engagement system (100) increments the crowd stipend by one as the group request entry will get removed from the table (and therefore revoked). At 1215, the crowd stipend stays unchanged when the first patient failed to resolve the cognitive response and the rational response. But if the first patient accepts the cognitive distortion and/or rational response from the teammate of the first patient, then the patient engagement system (100) decrements the crowd stipend by one.

At 1206, upon receiving the help request of the first patient, the patient engagement system (100) checks whether the ANT entry is still available in the group request. At 1207, when the ANT entry is not available in the group request, then the patient engagement system (100) increments the crowd stipend with one. At 1208, when the ANT entry is available in the group request, then the patient engagement system (100) responds to the teammate of the first patient. At 1209 and 1210, when the first patient likes the response of the teammate, then the patient engagement system (100) does not change the crowd stipend. At 1209 and 1211, when the first patient dislikes the response of the teammate, then the patient engagement system (100) increments the crowd stipend again by 1.

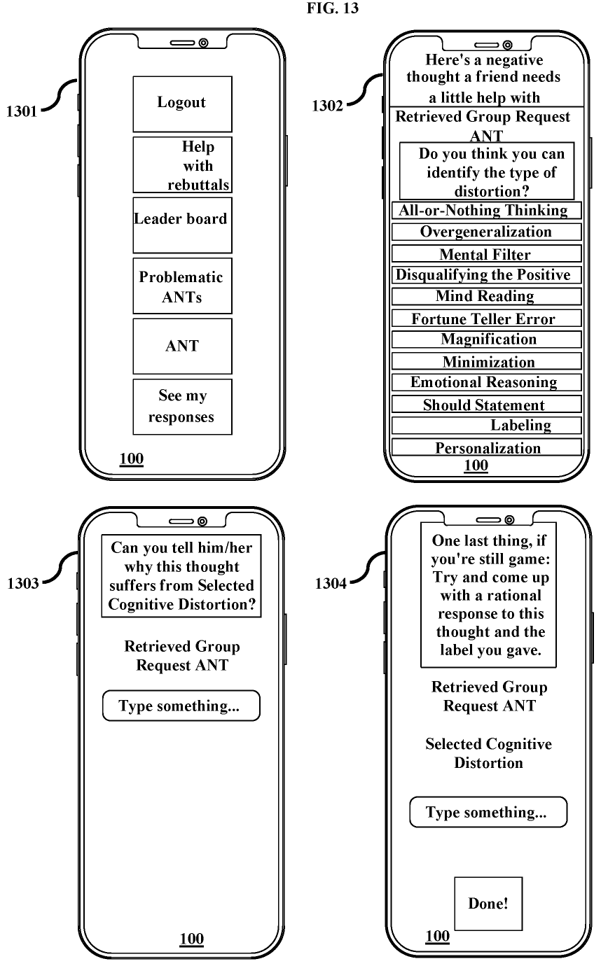

FIG. 13 illustrates an example scenario of obtaining the assistance of the teammate (i.e., second patient) for resolving the cognitive distortion of the rational response of the ANT of the first patient, according to embodiments as disclosed herein. Whenever the first patient has generated an ‘Ask a friend’ request, all the other team members (i.e., second patient, etc.) will get notified accordingly. Once the notification is received, the second patient will now be able to help the first patient by resolving the ANT and eventually will get the team score if the first patient approves the resolution of the ANT. This is possible if the second patient goes to the home screen of the patient engagement system (100) and clicks on the ‘Help with Rebuttals’ option in the screen of the patient engagement system (100) as shown in 1301 of FIG. 13. Once the second patient clicks this option ‘Help with Rebuttals’, the patient engagement system (100) directs the second patient to the screen as shown in 1302 of FIG. 13.

If the second patient has any requests from the teammates (i.e., first patient), then these requests will be displayed here but if there are no requests, the second patient will see the screen displaying ‘There are no requests from your teammates right now, check back later!’. If the second patient has any requests from the teammate (i.e., first patient), or if there are multiple potential teammate requests to be resolved, then the patient engagement system (100) selects a random group request ANT and retrieves for the second patient to help out the teammate. The second patient will not be able to see which of the teammates has requested the response for a particular ANT. The second patient can see the ANT of the teammate and will suggest cognitive distortion against the request.

After the selection of the cognitive distortion, now the first patient will be asked by the patient engagement system (100) to either provide reasoning to the selected cognitive distortion in the screen or to cancel as shown in 1303 of the FIG. 13. If the second patient selects the cancel option, then the patient engagement system (100) directs the second patient to the home screen and does not allocate any points to the second patient. The entry within the group request table will still be available to be answered by the teammates. Once the second patient has opened the teammate's request, no other teammate will be able to click and view this request for a period of time (e.g., 2 minutes). This means that only one teammate is allowed to respond to the generated teammate's request at a time.

If the second patient provides the reasoning ‘Rationale’ to select the cognitive distortion, then the patient engagement system (100) directs the second patient to the screen as shown in 1304 of the FIG. 13 where the first patient will be able to provide the rational response as well. If the second patient selects cancel, then the patient engagement system (100) directs the second patient to the home screen and does not provide any points to the second patient and the entry within the group request table will still be available to be answered by the teammates.

In the screen as shown in 1304 of the FIG. 13, the second patient is allowed to add the rational response in the input field provided against the suggested cognitive distortion and click on ‘Done’ afterward. The second patient is not allowed to click on ‘Done’ if the second patient hasn't provided the rational response. Once the second patient has clicked ‘Done’, then the patient engagement system (100) directs the second patient to the home screen and performs the steps listed below:
Store the ANT along with the request patient's username, the rational response, and the cognitive distortion in the group request database table.
Send a notification to the teammate who generated the request saying ‘Good news! Help has arrived!’

If the second patient is not able to provide the rational response, then the patient engagement system (100) directs the second patient to perform the untwist-your-thinking strategy activity. If the second patient is not able to generate the rational response using the untwist-your-thinking strategy activity, then the patient engagement system (100) directs the second patient to the home screen and does not provide any points to the second patient. The entry within the group request table will still be available to be answered by the teammate. Else, if the second patient provides the rational response using the untwist-your-thinking strategy activity, the patient engagement system (100) performs the steps listed below:
Stores the ANT along with the requested person's username, the rational response, the cognitive distortion, and the cognitive distortion rationale in the group request database table.
Send a notification to the teammate who generated the request saying ‘Good news! You have a response for a tricky ANT!’.

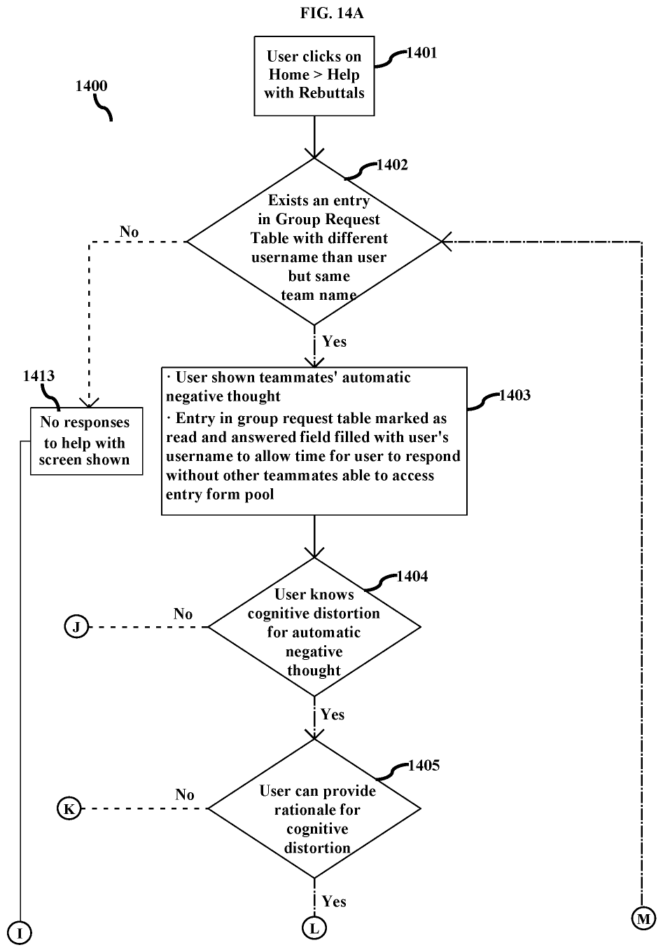

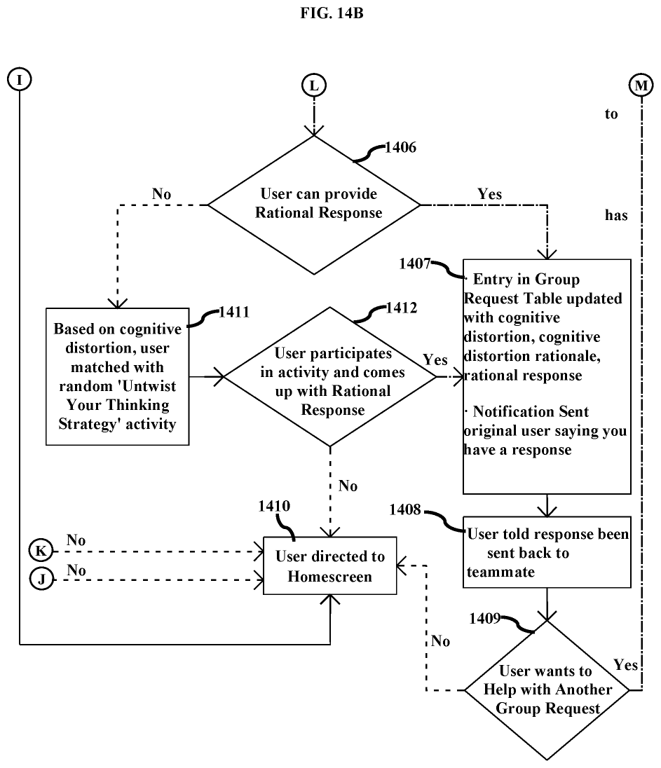

FIG. 14A to FIG. 14B (collectively referred to as FIG. 14) constitute a flow chart (1400) illustrating a method of handling the crowd stipend and providing the cognitive distortion and the rational response to the first patient, according to embodiments as disclosed herein. The flow chart (1400) includes the steps 1401-1413. At 1401 and 1405, upon clicking on the help with rebuttals option in the home screen by the second patient, the patient engagement system (100) checks whether the entry exists in the group request table with a different username than the second patient but the same team name. At 1413, when no entry exists in the group request table, then the patient engagement system (100) directs the second patient to the home screen at 1410. At 1403, when the entry exists in the group request table, then the patient engagement system (100) shows the teammate's ANT to the second patient.

Further, the patient engagement system (100) marks the entry in the group request table as read and fills an answered field with the second patient's username to allow time for the second patient to respond without other teammates being able to access the entry from the pool. At 1404, 1405 and 1406, when the second patient knows the cognitive distortion for the ANT and provides the rationale for the cognitive distortion, then the patient engagement system (100) checks whether the second patient can provide the rational response for the ANT. When the second patient does not know the cognitive distortion for the ANT or is unable to provide the rationale for the cognitive distortion, then the patient engagement system (100) directs the second patient to the home screen at step 1410.

At 1407, when the second patient provides the rational response for the ANT, then the patient engagement system (100) updates the entry in the group request table with the cognitive distortion, the cognitive distortion rationale, and the rational response. Further, the patient engagement system (100) sends the notification to the teammate indicating that the teammate has the response. At 1408, the patient engagement system (100) sends the response of the second patient to the teammate. At 1409, when the second patient wants to help with another group request, then the patient engagement system (100) continues to perform step 1402. When the second patient does not want to help with another group request, then the patient engagement system (100) continues to perform step 1410.

At 1411, when the second patient cannot provide the rational response for the ANT, then the patient engagement system (100) matches the second patient with the random untwist-your-thinking strategy activity based on the cognitive distortion. At 1412, when the second patient does not participate in the activity and/or does not come up with the rational response, then the patient engagement system (100) continues to perform the step 1410, whereas when the second patient participates in the activity and comes up with the rational response, then the patient engagement system (100) continues to perform the step 1407.

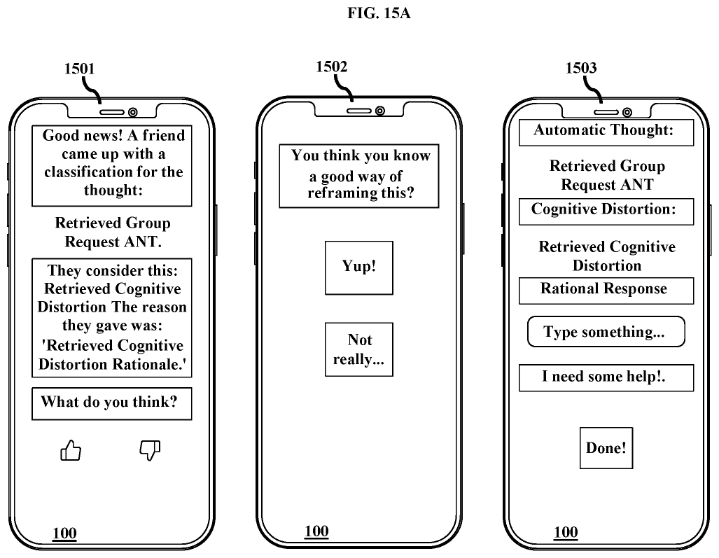

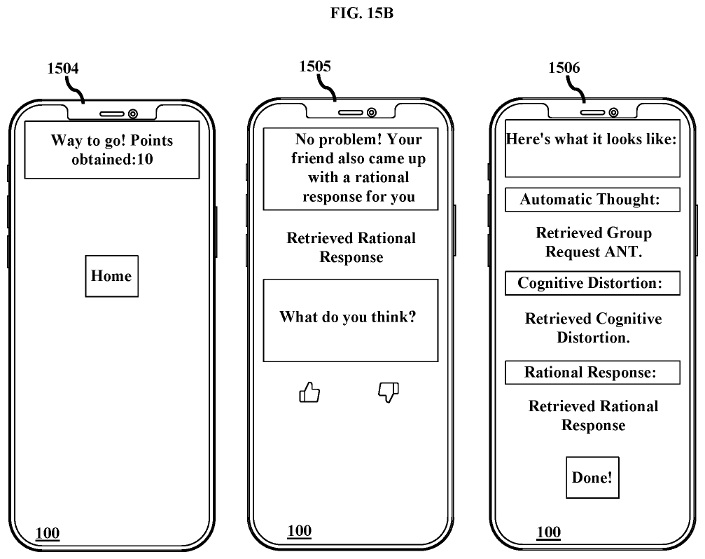

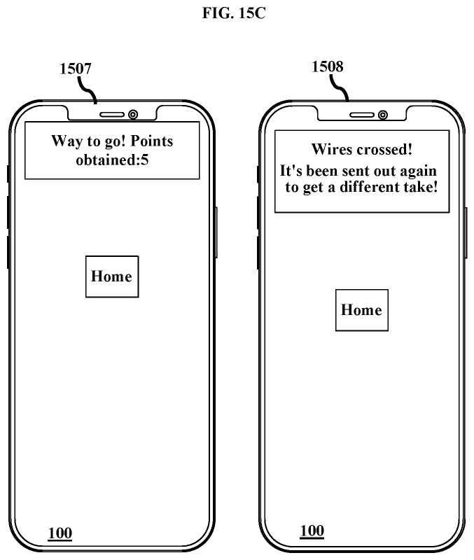

FIG. 15A to FIG. 15C (collectively referred to as FIG. 15) illustrates an example scenario of handling the cognitive distortion and/or the rational response provided by the second patient for the ANT of the first patient, according to embodiments as disclosed herein. Once the first patient generates any group request and this group request is sent to the group members and the group members respond by providing the cognitive distortion, the cognitive distortion rationale, and the rational response. This record will now be available to view by the first patient as shown in 1501 of FIG. 15, if the first patient clicks on the ‘See My Responses’ option on the home screen. The username of the second patient who created the response will not be shown, but the details provided by the second patient i.e., the cognitive distortion, the cognitive distortion rationale and the rational response will be available to be viewed by the first patient. If the first patient approves the cognitive distortion suggested by the second patient and likes the response, then the patient engagement system (100) displays the screen shown in 1502 of FIG. 15.

Further, the patient engagement system (100) asks the first patient whether the first patient knows the rational response against the cognitive distortion or not. If the first patient selects ‘Yup’ then the first patient will be taken to another screen as shown in 1503 of FIG. 15 by the patient engagement system (100), where the first patient will be able to provide the rational response. Further, the first patient is allowed to add the rational response or ask for help from the application and will get matched to the untwist-your-thinking strategy. If the first patient enters the rational response and clicks ‘Done’, then the patient engagement system (100) performs the list of steps given below:
Allocate 10 points to the first patient, and 15 points to the second patient who suggested the resolution.
Send the notification saying ‘Congratulations! You have been awarded 15 points for helping out.’ to the second patient
Allocate 25 points to the team.
Remove the ANT from the group request table
Remove the ANT from the problematic request table
Store the ANT entry in the triple-column table with the respective accepted cognitive distortion and rational response.

Further, the patient engagement system (100) displays the screen as shown in 1504 of FIG. 15 to the first patient. The first patient will be able to go to the home screen as well. If the first patient likes the cognitive distortion suggested by the second patient and the first patient doesn't know the rational response, then the patient engagement system (100) displays the screen as shown in 1505 of the FIG. 15 (where the retrieved rational response is the rational response submitted by the teammate) to the first patient. Further, the patient engagement system (100) displays the rational response suggested by the teammate to the first patient. If the first patient likes the suggested rational response, then the patient engagement system (100) displays a summary screen of the ANT, the suggested ‘Cognitive Distortion’, and the suggested ‘Rational Response’ as shown in 1506 of the FIG. 15 the first patient. Once the first patient clicks ‘Done’ on this summary screen, then the patient engagement system (100) performs the list of steps given below:
Allocate 5 points to the first patient.
Allocate 25 points to the first patient's team.
Allocate 20 points to the second patient.
Send the notification saying ‘Congratulations! You have been awarded 20 points for helping out.’ to the second patient who resolved the ANT.
Remove the ANT record from the problematic ANTs table as well as the group request table.

Further, the first patient is allowed to move to the home screen as well as shown in 1507 of FIG. 15. If the first patient likes the cognitive distortion and dislikes the rational response suggested by the second patient, then the patient engagement system (100) directs the first patient to the screen of the untwist-your-thinking strategy. If the first patient participates in this activity and comes up with the rational response, then the patient engagement system (100) performs the list of steps given below:
Allocate 10 points to the first patient, 15 points to the second patient who suggested the resolution.
Send the notification stating ‘Congratulations! You have been awarded 15 points for helping out.’ to the second patient.
Allocate 25 points to the team.
Remove the ANT from the group request table
Remove the ANT from the problematic ANT table.
Store the ANT entry in the triple-column table with the respective accepted cognitive distortion and rational response

Further, the first patient is allowed to move to the home screen. If the first patient doesn't participate in the activity, then the patient engagement system (100) moves the generated ANT to the problematic ANTs table and remains in the group request section (after removing any unhelpful fields that were input by the responding teammate i.e., cognitive distortion, cognitive distortion rationale, rational response). In other words, the ANT request remains to be resolved by any other teammate and the notification will be sent, etc.

If the first patient dislikes the cognitive distortion suggested by the group member, then the patient engagement system (100) performs the list of steps given below:
Send the ANT in the group request table and display in the ‘help with rebuttals’ section to the other group members.
No points will be allocated to the first patient.
The ANT will remain in the problematic thoughts table (110).
Send a notification saying, ‘Your team member needs help.’ to the second patient.

Further, the patient engagement system (100) displays the screen as shown in 1508 of FIG. 15.

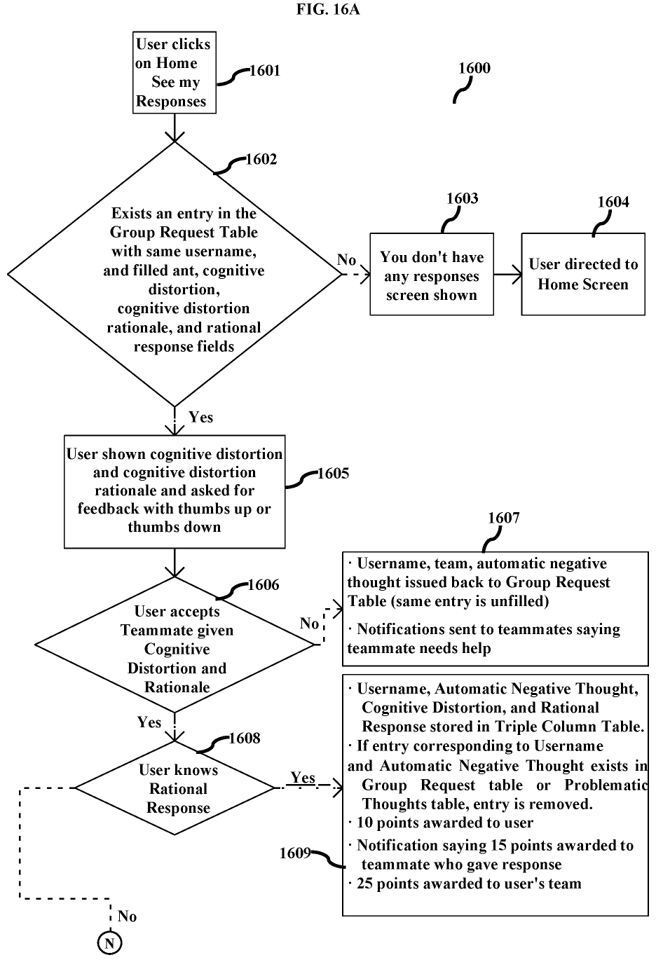

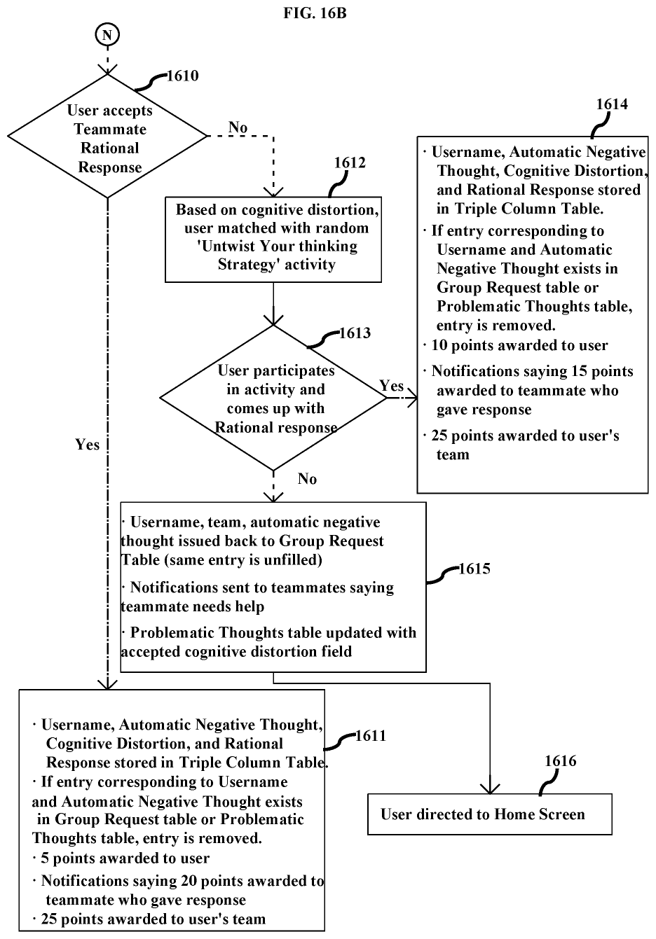

FIG. 16A to FIG. 16B (collectively referred to as FIG. 16) is a flow chart (1600) illustrating a method of handling the cognitive distortion and/or the rational response provided by the second patient for the ANT of the first patient, according to embodiments as disclosed herein. The flow chart (1600) includes the steps 1601-1616. At 1601 and 1602, upon clicking on the home screen to view the responses of the teammates (second patient) by the first patient, the patient engagement system (100) checks whether the entry in the group request table is existing with the same username, and ANT, the cognitive distortion, the cognitive distortion rationale, and the rational response fields are filled. At 1603 and 1604, when the entry in the group request table is not existing, then the patient engagement system (100) displays to the first patient that the first patient doesn't have any responses to show and then directs the first patient to the home screen.

At 1605, the patient engagement system (100) displays the cognitive distortion and the cognitive distortion rationale to the first patient and asks the first patient for feedback with thumbs up or thumbs down. At 1616, the patient engagement system (100) checks whether the first patient has accepted the teammate given cognitive distortion and rationale. At 1607, when the first patient has not accepted the teammate given cognitive distortion and rationale, then the patient engagement system (100) records the username, the team, the ANT issued back to the group request table (same entry is unfilled) and sends notifications to the teammates saying the first patient needs help.

At 1608, when the first patient has accepted the teammate given cognitive distortion and rationale, then the patient engagement system (100) asks the first patient to provide the rational response. At 1609, when the first patient has provided the rational response to the patient engagement system (100), then the patient engagement system (100) fills the username, the ANT, the cognitive distortion, and the rational response stored in the triple-column table. If the entry corresponding to the username, and the ANT exist in the group request table or the problematic thoughts table (110), then the patient engagement system (100) removes that entry. Further, the patient engagement system (100) awards 10 points to the first patient, and 25 points to the team of the first patient. Further, the patient engagement system (100) sends a notification to the first patient saying 15 points awarded to the teammate who has given the response.

At 1610 and 1611, when the first patient accepts the teammate rational response, then the patient engagement system (100) fills in the username, the ANT, the cognitive distortion, and the rational response stored in the triple-column table. If the entry corresponding to the username, the ANT exists in the group request table or the problematic thoughts table (110), then the patient engagement system (100) removes that entry. Further, the patient engagement system (100) awards 5 points to the first patient, and 25 points to the team of the first patient. Further, the patient engagement system (100) sends a notification to the first patient saying 20 points are awarded to the teammate who has given the response.

At 1610 and 1612, when the first patient doesn't accept the teammate's rational response, then the patient engagement system (100) matches the first patient with the random untwist-your-thinking strategy activity based on the cognitive distortion. At 1613 and 1614, when the first patient participates in the activity and comes up with the rational response, then the patient engagement system (100) fills the username, the ANT, the cognitive distortion, and the rational response stored in the triple-column table. If the entry corresponding to the username, the ANT exists in the group request table or the problematic thoughts table (110), then the patient engagement system (100) removes that entry. Further, the patient engagement system (100) awards 10 points to the first patient, and 25 points to the team of the first patient. Further, the patient engagement system (100) sends a notification to the first patient saying 15 points are awarded to the teammate who has given the response.

At 1613 and 1615, when the first patient does not participate in the activity and/or does not come up with the rational response, then the patient engagement system (100) fills the username, the ANT, issued back to the group request table (same entry is unfilled), sends the notifications to the teammates saying the first patient needs help and updates the problematic thoughts table (110) with the accepted cognitive distortion field. At 1616, the patient engagement system (100) further directs the first patient to the home screen.

If the entry corresponding to the username, the ANT exists in the group request table or problematic thoughts table (110), then the patient engagement system (100) removes that entry. Further, the patient engagement system (100) awards 10 points to the first patient, and 25 points to the team of the first patient. Further, the patient engagement system (100) sends a notification to the first patient saying 15 points are awarded to the teammate who has given the response.

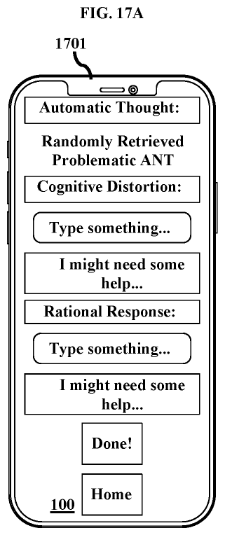

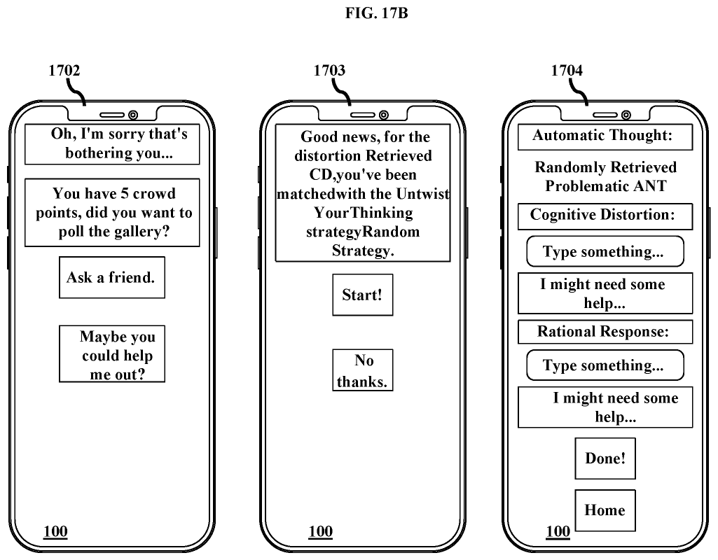

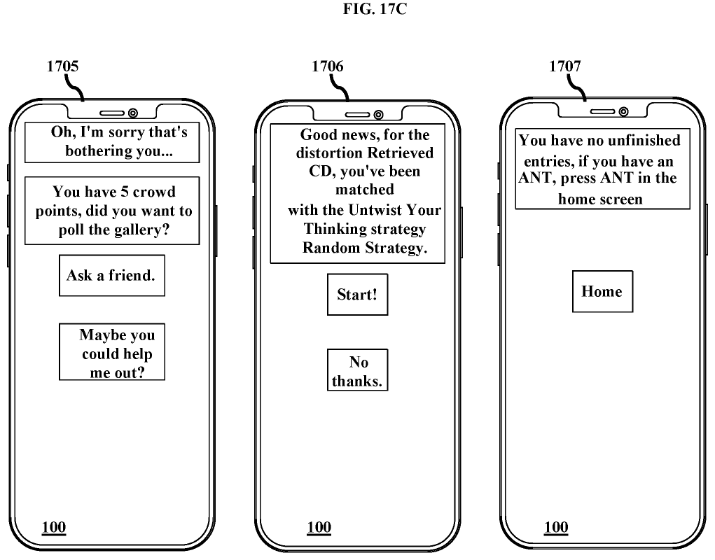

FIG. 17A to FIG. 17C (collectively referred to as FIG. 17) illustrates an example scenario of handling the problematic ANT by the patient engagement system (100), according to embodiments as disclosed herein. When the first patient cannot resolve the ANT or with the help of the application & other team members, this ANT is sent to the problematic ANTs table. Any ANT having no cognitive distortion, or no rational response, or no cognitive distortion and rational response is a part of the problematic ANT table. Once the first patient goes to the home screen and clicks on the problematic ANT table, then the patient engagement system (100) displays unresolved ANTs to the first patient as shown in 1701 of FIG. 17. Further, the first patient is allowed to see a randomly retrieved (if multiple exist) problematic ANT with respective cognitive distortion (if available).

If cognitive distortion is available in the problematic ANT table, the first patient is allowed to edit it. Also, the first patient will have an option of ‘I think I might need some help’ below the cognitive distortion as well as below the rational response field. If the first patient clicks on the ‘I think I might need some help button below the cognitive distortion field, the patient engagement system (100) directs the first patient to the screen as shown in 1702 of the FIG. 17, where the first patient can either ‘Ask a Friend’ or take help from the application by clicking on ‘Maybe you could help me out’ option.

If the first patient clicks on ‘I might need some help’ below the rational response field, the patient engagement system (100) checks whether the cognitive distortion field is empty. If it's empty, the patient engagement system (100) prompts the first patient to add cognitive distortion first. If the cognitive distortion field is not empty, then the patient engagement system (100) directs the first patient to perform the ‘Untwist-your-thinking Strategy’ activity which will be matched as shown in 1703 of FIG. 17. If the first patient adds the rational response in the problematic ANT with the cognitive distortion being available, then the patient engagement system (100) performs the list of steps given below:
Store the resolved ANT having ‘Username, ANT, Cognitive Distortion and Rational Response’ in the triple-column table.
Allocate 10 points to the first patient and 10 points to the team.

Whenever the first patient generates a request to ask for help from the application, this ANT record will move to the problematic ANTs table as well as to the group request table. Now when the ANT is resolved, this ANT record will get removed from both the sections i.e., the problematic ANTs table and the group requests ANTs table. The first patient will only be able to achieve this step if the first patient provides cognitive distortion. If the first patient does not provide cognitive distortion, the patient engagement system (100) asks the first patient ‘Please provide Cognitive Distortion’ to continue.

If the cognitive distortion is unavailable, the first patient will have empty fields for the cognitive distortion and the rational response available with the ‘I think I might need some help’ button below both of them respectively as shown in 1704 of the FIG. 17. The first patient will have an option of ‘I think I might need some help’ below the cognitive distortion as well as below the rational response field. If the first patient clicks on the ‘I think I might need some help’ button below the cognitive distortion field, the patient engagement system (100) directs the first patient to the screen as shown in 1705 of the FIG. 17, where the first patient can either ‘Ask a Friend’ or take help from the application by clicking on ‘Maybe you could help me out’ option in this screen.

If the first patient clicks on ‘I might need some help’ below the rational response field, the patient engagement system (100) checks whether the cognitive distortion field is empty. If it's empty, the patient engagement system (100) prompts the first patient to add cognitive distortion. If the cognitive distortion field is not empty, then the patient engagement system (100) directs the first patient to perform the random untwist-your-thinking strategy activity which is selected according to the recorded cognitive distortion as shown in 1706 of the FIG. 17. If the first patient adds the rational response in the problematic ANT with the cognitive distortion being available, the patient engagement system (100) performs the list of steps given below:
Store the resolved ANT having ‘Username, ANT, cognitive distortion and rational response’ into the triple-column table.
Allocate 10 points to the first patient and 10 points to the team.

As mentioned, whenever the first patient generates the request to ask for help from the application, the patient engagement system (100) moves this ANT record to the problematic ANTs table. When this ANT is resolved, the patient engagement system (100) removes this ANT record from the problematic ANTs table and the group request table if the ANT record exists in these tables.

The first patient will only be able to achieve this step if the first patient has provided the cognitive distortion. If the first patient does not provide cognitive distortion, then the patient engagement system (100) asks the first patient ‘Please provide Cognitive Distortion’ to continue. If there are no problematic thoughts associated with the first patient's username in the problematic thoughts table (110), then the patient engagement system (100) displays the screen as shown in 1707 of FIG. 17.

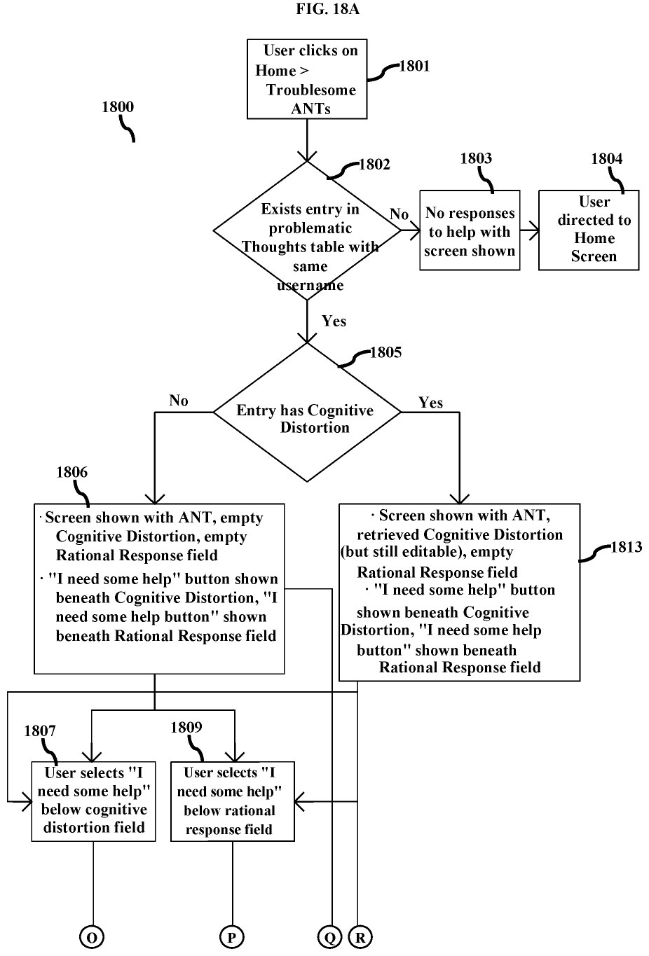

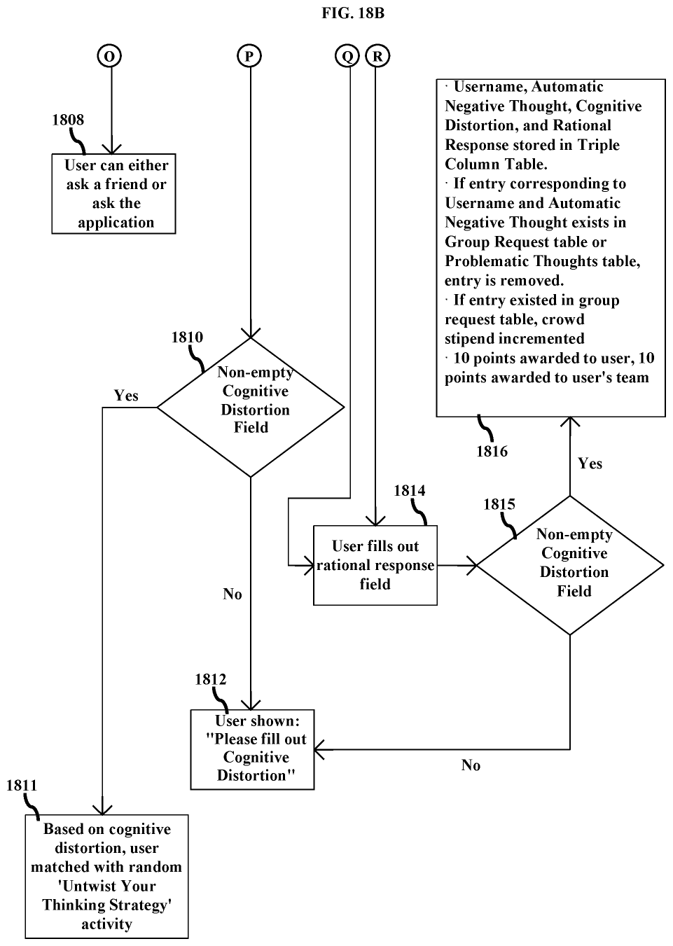

FIG. 18A to FIG. 18B (collectively referred to as FIG. 18) constitute a flow chart (1800) illustrating a method of the problematic ANT by the system (100), according to embodiments as disclosed herein. The flow chart (1800) includes the steps 1801-1813. At 1801, the patient engagement system (100) detects first patient's input on the troublesome ANTs in the home screen. At 1802, the patient engagement system (100) checks whether the entry is existing in the problematic thoughts table (110) with same username. At 1803 and 1804, when the entry is not existing in the problematic thoughts table (110) with the same username, then the patient engagement system (100) does not find the responses to help the first patient with the screen shown and directs the first patient to the home screen. At 1805, when the entry is existing in the problematic thoughts table (110) with same username, then the patient engagement system (100) checks whether the entry has the cognitive distortion. At 1806, when the entry has the cognitive distortion, then the patient engagement system (100) displays the ANT, an empty cognitive distortion field, an empty rational response field, a “I need some help” button shown beneath the empty cognitive distortion field, and another “I need some help” button shown beneath the empty rational response field. At 1807 and 1808, when the first patient selects the “I need some help” button below the empty cognitive distortion field, then the patient engagement system (100) allows the first patient to either ask for assistance of the friend or ask the application.

At 1809 and 1810, when the first patient selects the “I need some help” button below the empty rational response field, then the patient engagement system (100) checks whether the cognitive distortion field is non-empty. At 1811, when the cognitive distortion field is non-empty, then the patient engagement system (100) matches the first patient with the random untwist-your-thinking strategy activity based on the cognitive distortion. At 1812, when the cognitive distortion field is empty, then the patient engagement system (100) prompts the first patient to fill out the cognitive distortion field.

At 1813, when the entry hasn't the cognitive distortion, then the patient engagement system (100) displays the ANT, the retrieved cognitive distortion (but still editable), the empty rational response field, the “I need some help” button shown beneath the cognitive distortion field, and another “I need some help” button shown beneath the empty rational response field. From step 1813, the patient engagement system (100) further performs steps 1807 or 1809 or 1814. From step 1806, the patient engagement system (100) may further perform step 1814. At 1814, the patient engagement system (100) allows the first patient to fill out the rational response field. At 1815, the patient engagement system (100) checks whether the cognitive distortion field is non-empty. At 1816, when the cognitive distortion field is non-empty, then the patient engagement system (100) enters the username, the ANT, the cognitive distortion, and the rational response in the triple-column table. If an entry corresponding to the username and the ANT exists in the Group Request table or the Problematic thoughts table (110), then the patient engagement system (100) removes that entry. If the entry existed in the group request table, then the patient engagement system (100) increments the crowd stipend, and awards 10 points to the first patient, and 10 points to the team. When the cognitive distortion field is empty, then the patient engagement system (100) performs step 1812.

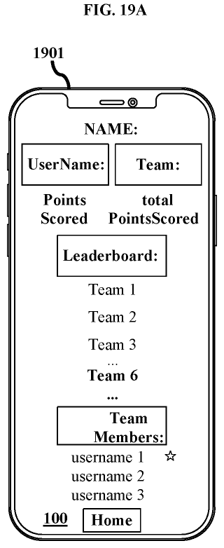

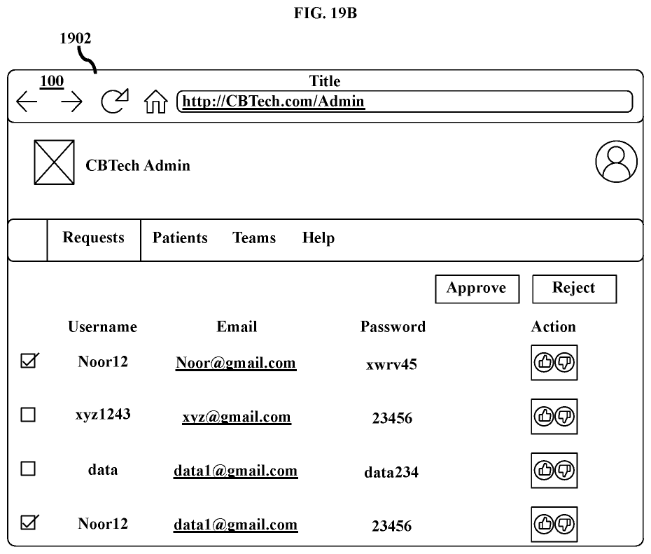

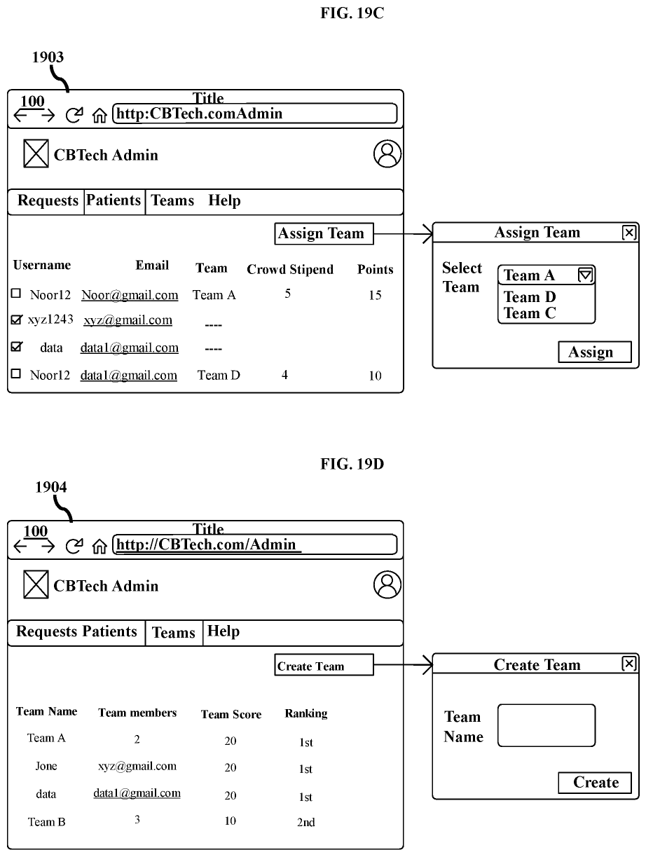

FIG. 19A to FIG. 19D (collectively referred to as FIG. 19) illustrates an example scenario of displaying the leaderboard, and the admin portal by the patient engagement system (100), according to embodiments as disclosed herein. From the home screen, the patient will have the option to view the ‘Leaderboard’. In the ‘Leaderboard’, the patient's scores along with the username and the following team information will be displayed as shown in 1901 of the FIG. 19:
Team Name
Team members (usernames)
Team Score
Team Ranking

This screen shown in 1901 of FIG. 19 will also display other teams' rankings as well (top 3 teams). The group leader will have a crown or star marked next to the first patient's username under the team members list. If the patient clicks on ‘Home’, the patient will land on the home screen.

The patient engagement system (100) will have the admin portal, this admin portal will be used to manage patients' data, and teams and approve/disapprove signup requests. The following are the different sections of the admin portal.

Signup Request (as shown in 1902 of FIG. 19): Whenever any new patient generates a signup request from the application, this request will flow into the admin portal from where the admin will be able to approve or disapprove the request. In the signup request section, the admin will be able to perform the following functions:
View Signup requests along with the patient's name, email, and password.
Approve/Disapprove requests individually
Approve/Disapprove request by multi-selection.

Once any signup request is approved, this data will go into the patient's table and the patient will also receive an approval email to sign into the application.

Patients (shown in 1903 of FIG. 19): Once the signup request is approved, the patient's data will flow in the patients' table with no team assigned yet. The admin will be able to assign a team to this patient from here and see the first patient's information.

Teams (shown in 1904 in FIG. 19): The teams' section will display all the team-related information, the patient will be able to view team members' information as well as create new teams from here as well. Only after a patient is assigned a team will get a notification allowing the patient to login to the application.

The embodiments disclosed herein can be implemented using at least one hardware device and performing network management functions to control the elements.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the scope of the claims as described herein.

We claim:

1. A patient engagement method comprising:
receiving, by a patient engagement system (100), an input from a first patient from a plurality of patients, wherein the input indicates that the first patient has an Automatic Negative Thought (ANT);
displaying, by the patient engagement system (100), a first option to participate in a triple-column associated with the ANT of the first patient, a second option for assistance of at least one second patient of the plurality of patients, and a third option for assistance of an application;
detecting, by the patient engagement system (100), one of the first option, the second option and the third option selected by the first patient; and
performing, by the patient engagement system (100), one of:
when the first option is selected by the first patient, displaying the triple-column, receiving an input corresponding the ANT, a cognitive distortion and a rational response in the triple-column from the first patient and allocating a score to the first patient based on the input provided by the first patient,
when the second option is selected by the first patient, sending a cognitive distortion and rational response request to the at least one second patient, receiving the cognitive distortion and the rational response for the ANT from the at least one second patient, and allocating a score to the at least one second patient and the first patient upon acceptance of the first patient for the cognitive distortion and the rational response for the ANT received from the at least one second patient, and
when the third option is selected by the first patient, predicting the cognitive distortion and the rational response for the ANT using a Machine Learning (ML) model (116) associated with the application, and allocating a score to the first patient upon acceptance of the first patient for the cognitive distortion and the rational response for the ANT predicted by the ML model (116).

2. The method as claimed in claim 1, wherein allocating the score to the at least one second patient and the first patient upon acceptance of the first patient for the cognitive distortion and the rational response for the ANT received from the at least one second patient comprising:
adding the score to a previously allocated score of the at least one second patient, and the first patient upon acceptance of the first patient for the cognitive distortion and the rational response for the ANT of the first patient received from the at least one second patient.

3. The method as claimed in claim 1, wherein allocating the score to the first patient upon acceptance of the first patient for the cognitive distortion and the rational response for the ANT predicted by the ML model (116) comprises:
adding the score to a previously allocated score of the first patient upon acceptance of the first patient for the cognitive distortion and the rational response predicted by the ML model (116) for the ANT of the first patient.

4. The method as claimed in claim 1, wherein sending a cognitive distortion and rational response request to the at least one second patient comprises:
determining whether a crowd stipend of the first patient meets a crowd stipend threshold;
sending the cognitive distortion and rational response request to the at least one second patient when the crowd stipend of the first patient meets the crowd stipend threshold; and
decrementing the crowd stipend of the first patient.

5. The method as claimed in claim 1, wherein receiving, by the patient engagement system (100), the cognitive distortion and the rational response for the ANT from the at least one second patient, comprises:
receiving, by the patient engagement system (100), the cognitive distortion for the ANT and a rationale for selecting the cognitive distortion from the at least one second patient;
displaying, by the patient engagement system (100), an untwist-your-thinking strategy activity to be performed by the at least one second patient based on the cognitive distortion when only the cognitive distortion for the ANT and a rationale for selecting the cognitive distortion is received from the at least one second patient; and
receiving, by the patient engagement system (100), the rational response for the ANT upon performing the untwist-your-thinking strategy activity by the at least one second patient.

6. The method as claimed in claim 1, wherein acceptance of the first patient for the cognitive distortion and the rational response of the ANT received from the at least one second patient or predicted by the ML model (116) comprises:
sending, by the patient engagement system (100), the cognitive distortion and the rational response for the ANT to the first patient;
receiving, by the patient engagement system (100), an input indicating acceptance of the cognitive distortion and rejection of the rational response by the first patient;
displaying, by the patient engagement system (100), an untwist-your-thinking strategy activity to be performed by the first patient based on the input and the cognitive distortion; and
receiving, by the patient engagement system (100), the rational response for the ANT upon performing the untwist-your-thinking strategy by the first patient.

7. The method as claimed in claim 1, wherein the method comprises:
receiving, by the patient engagement system (100), the score allocated to each patient of the plurality of patients, wherein the plurality of patients is associated with a group;
assigning, by the patient engagement system (100), a score to the group by combining the score of each patient of the plurality of patients associated with the group;
storing, by the patient engagement system (100), the score of the group and score allocated to each patient of the plurality of patients in the patient engagement system (100) into a memory (120); and
displaying, by the patient engagement system (100), the score and a rank of each group.

8. The method as claimed in claim 7, wherein the method comprises:
allocating, by the patient engagement system (100), a score to the first patient and the group proportional to an engagement of the first patient with the patient engagement system (100), and a therapist of the first patient.

9. The method as claimed in claim 1, wherein the method comprises:
detecting, by the patient engagement system (100), a rejection of the first patient on the cognitive distortion and the rational response for the ANT received from the ML model (116) or the at least one second patient; and
storing, by the patient engagement system (100), the ANT to a problematic thoughts database (110).

10. The method as claimed in claim 9, wherein the method comprises:
displaying, by the patient engagement system (100), the ANT in the problematic thoughts database (110) to a therapist during an in-person group therapy session or based on a user input; and
receiving, by the patient engagement system (100), the cognitive distortion and the rational response for the ANT from the therapist.

11. The method as claimed in claim 1, wherein the method comprises:
storing, by the patient engagement system (100), the cognitive distortion and the rational response for the ANT into a memory (120).

12. The method as claimed in claim 1, wherein the predicting the cognitive distortion and the rational response for the ANT using the ML model (116) associated with the application comprises:
retrieving, by the patient engagement system (100), a previously stored ANT similar to the ANT using the ML model (116), and a cognitive distortion and a rational response of the previously stored ANT from a memory (120).

13. The method as claimed in claim 1, wherein the predicting the cognitive distortion and the rational response for the ANT using the ML model (116) associated with the application comprises:
assigning, by the patient engagement system (100), a cognitive distortion for an unrecognized ANT.

14. A patient engagement system (100) comprising:
a memory (120) comprising information of a plurality of patients; and
a processor (150) coupled to the memory (120), wherein the processor (150):
receives an input from a first patient from the plurality of patients, wherein the input indicates that the first patient has an Automatic Negative Thought (ANT);
displays a first option to participate in a triple-column associated with the ANT of the first patient, a second option for assistance of at least one second patient of the plurality of patients, and a third option for assistance of an application;
detects one of the first option, the second option and the third option selected by the first patient; and
performs one of:
when the first option is selected by the first patient, displaying the triple-column, receiving an input corresponding the ANT, a cognitive distortion and a rational response in the triple-column from the first patient and allocating a score to the first patient based on the input provided by the first patient,
when the second option is selected by the first patient, sending a cognitive distortion and rational response request to the at least one second patient, receiving the cognitive distortion and the rational response for the ANT from the at least one second patient, and allocating a score to the at least one second patient and the first patient upon acceptance of the first patient for the cognitive distortion and the rational response for the ANT received from the at least one second patient, and
when the third option is selected by the first patient, predicting the cognitive distortion and the rational response for the ANT using a Machine Learning (ML) model (116) associated with the application, and allocating a score to the first patient upon acceptance of the first patient for the cognitive distortion and the rational response for the ANT predicted by the ML model (116).

15. The patient engagement system (100) as claimed in claim 14, wherein allocating the score to the at least one second patient and the first patient upon acceptance of the first patient for the cognitive distortion and the rational response for the ANT received from the at least one second patient comprising:
adding the score to a previously allocated score of the at least one second patient, and the first patient upon acceptance of the first patient for the cognitive distortion and the rational response for the ANT of the first patient received from the at least one second patient.

16. The patient engagement system (100) as claimed in claim 14, wherein allocating the score to the first patient upon acceptance of the first patient for the cognitive distortion and the rational response for the ANT predicted by the ML model (116) comprises:
adding the score to a previously allocated score of the first patient upon acceptance of the first patient for the cognitive distortion and the rational response predicted by the ML model (116) for the ANT of the first patient.

17. The patient engagement system (100) as claimed in claim 14, wherein sending a cognitive distortion and rational response request to the at least one second patient comprises:
determining whether a crowd stipend of the first patient meets a crowd stipend threshold;
sending the cognitive distortion and rational response request to the at least one second patient when the crowd stipend of the first patient meets the crowd stipend threshold; and
decrementing the crowd stipend of the first patient.

18. The patient engagement system (100) as claimed in claim 14, wherein receiving the cognitive distortion and the rational response for the ANT from the at least one second patient, comprises:
receiving the cognitive distortion for the ANT and a rationale for selecting the cognitive distortion from the at least one second patient;
displaying an untwist-your-thinking strategy activity to be performed by the at least one second patient based on the cognitive distortion when only the cognitive distortion for the ANT and a rationale for selecting the cognitive distortion is received from the at least one second patient; and
receiving the rational response for the ANT upon performing the untwist-your-thinking strategy by the at least one second patient.

19. The patient engagement system (100) as claimed in claim 14, wherein acceptance of the first patient for the cognitive distortion and the rational response of the ANT received from the at least one second patient or predicted by the ML model (116) comprises:
sending the cognitive distortion and the rational response for the ANT to the first patient;
receiving an input indicating acceptance of the cognitive distortion and rejection of the rational response by the first patient;
displaying an untwist-your-thinking strategy activity to be performed by the first patient based on the input and the cognitive distortion; and
receiving the rational response for the ANT upon performing the untwist-your-thinking strategy by the first patient.

20. The patient engagement system (100) as claimed in claim 14, wherein the processor (150):
receives the score allocated to each patient of the plurality of patients, wherein the plurality of patients is associated with a group;
assigns a score to the group by combining the score of each patient of the plurality of patients associated with the group;
stores the score of the group and score allocated to each patient of the plurality of patients into a memory (120); and
displays the score and a rank of each group.

21. The patient engagement system (100) as claimed in claim 20, wherein the processor (150):
allocates a score to the first patient and the group proportional to an engagement of the first patient with the patient engagement system (100), and a therapist of the first patient.

22. The patient engagement system (100) as claimed in claim 14, wherein the processor (150):
detects a rejection of the first patient on the cognitive distortion and the rational response for the ANT received from the ML model (116) or the at least one second patient; and
stores the ANT to a problematic thoughts database (110).

23. The patient engagement system (100) as claimed in claim 22, wherein the processor (150):
displays the ANT in the problematic thoughts database (110) to a therapist during an in-person group therapy session or based on a user input; and
receives the cognitive distortion and the rational response for the ANT from the therapist.

24. The patient engagement system (100) as claimed in claim 14, wherein the processor (150):
stores the cognitive distortion and the rational response for the ANT into a memory (120).

25. The patient engagement system (100) as claimed in claim 14, wherein the predicting the cognitive distortion and the rational response for the ANT using the ML model (116) associated with the application comprises:
retrieving a previously stored ANT similar to the ANT using the ML model (116), and a cognitive distortion and a rational response of the previously stored ANT from a memory (120).

26. The patient engagement system (100) as claimed in claim 14, wherein the predicting the cognitive distortion and the rational response for the ANT using the ML model (116) associated with the application comprises:
assigning a cognitive distortion for an unrecognized ANT.
